(19) United States
(12) Patent Application Publication
(10) Pub. No.: US 2023/0349887 A1
(43) Pub. Date: Nov. 2, 2023

(54) METHOD OF USING EXTRACELLULAR VESICLES TO DETECT COMPLEMENT ACTIVATION, AND USES THEREOF TO ASSESS AND/OR MONITOR TREATMENT OF A COMPLEMENT-MEDIATED DISEASE

(71) Applicant: Alexion Pharmaceuticals, Inc., Boston, MA (US)

(72) Inventors: Ellen E. Millman, Southbury, CT (US); Tobin J. Cammett, Southbury, CT (US)

(73) Assignee: Alexion Pharmaceuticals, Inc., Boston, MA (US)

(21) Appl. No.: 17/923,148

(22) PCT Filed: May 13, 2021
(86) PCT No.: PCT/US2021/032241

§ 371 Date: Nov. 3, 2022

Related U.S. Application Data
(60) Provisional application No. 63/025,557, filed on May 15, 2020.

(51) Int. Cl. G01N 33/50; G01N 33/53; G01N 33/68

(52) U.S. Cl. CPC G01N 33/5076; G01N 33/5308; G01N 33/6863; G01N 2333/4716; G01N 2333/705

(57) ABSTRACT

Disclosed herein are methods of detecting complement activity in a biological sample. The disclosure also relates to methods for diagnosis or prognostic assessment of a complement-mediated disease in a subject and methods for monitoring response to treatment of a complement-mediated disease with a complement modulator in a subject.

### CROSS REFERENCE TO RELATED APPLICATIONS

This application claims priority to, and the benefit of, U.S. Provisional Application No. 63/025,557, filed on May 15, 2020, the entire contents which are incorporated herein by reference.

### FIELD

The present disclosure relates to methods of detecting complement activation in a biological sample. The disclosure also relates to methods for diagnosis or prognostic assessment of a complement-mediated disease in a subject, and methods for monitoring response during and after treatment of a complement-mediated disease with a complement modulator.

### BACKGROUND

The complement system is part of the innate immune system and acts in conjunction with other immunological systems of the body to defend against intrusion of cellular and viral pathogens. There are at least 25 proteins in the complement pathway, which are found as a complex collection of circulating plasma proteins and cell membrane cofactors. The plasma proteins make up about 10% of the globulins in vertebrate sera. The complement proteins circulate in the blood as inactive precursors and, when stimulated by one of several triggers, proteases in the system cleave specific proteins to release cytokines and initiate an amplifying cascade of further cleavages.

Complement components achieve their immune defensive functions by activating an intricate series of cell surface and fluid-phase interactions involving precise enzymatic cleavages and plasma membrane binding events. The resulting complement cascade leads to the production of products with opsonic, immunoregulatory, and lytic functions.

The complement cascade progresses via the classical pathway, the alternative pathway, or the lectin pathway. These pathways share many components, and while they differ in their initial steps, they converge and share the same “terminal complement” components (C5 through C9) responsible for the activation and destruction of target cells.

The classical pathway (CP) is typically initiated by antibody recognition of, and binding to, an antigenic site on a target cell. The alternative pathway (AP) is antibody independent and capable of autoactivation by certain molecules on pathogen surfaces. Additionally, the lectin pathway is typically initiated with binding of mannose-binding lectin (MBL) to high mannose substrates. These pathways converge at the point where complement component C3 is cleaved by an active protease to yield C3a and C3b. Other pathways activating complement attack can act later in the sequence of events leading to various aspects of complement function.

The complement system plays a vital role within the human body to fight diseases, and measurement of components in the complement system can be useful for diagnosis and/or prognosis of disease, as well as for monitoring response to treatment of a complement-mediated disease.

Tissue biopsy provides definitive clinical evidence for most disease diagnoses and can be a direct way to confirm a role for complement in disease pathogenesis. However, biopsies are painful and expensive, and there are risks associated with the procedure. A repeat tissue biopsy is rarely done. Therefore, monitoring longitudinal response to treatment via multiple local tissue biopsies is not possible.

Extracellular vesicles (EV) are small membrane-bound, enveloped particles (30-100 nm) made by cells. They are released from the plasma membrane (PM) of the parent cell, and contain functional membrane and cytosolic proteins, lipids, and RNAs. Other terms for EVs include: microvesicles, ectosomes, shedding vesicles, microparticles, and exosomes. The EV external membrane contains EV-specific protein markers and PM markers specific to the parent cell. The orientation of the EV membrane protein is the same as in the parent PM. Extracellular vesicles carry canonical EV markers such as CD9, CD63, or CD81, which are members of the tetraspanin superfamily of proteins. Tetraspanins are among the most abundant membrane proteins of EVs. Some EV also carry complement regulators on their surfaces, such as CD55 and CD59. Healthy urine contains approximately 109 urinary EVs/mL (uEV/mL), which originate predominantly from kidney, urinary tract epithelium, and (in males) the reproductive tract. Cells under stress will increase EV production.

A current unmet need in the field of complement diagnostics and therapy is a sensitive, specific and non-invasive clinical test for measuring localized tissue deposition of terminal complement complex. A non-invasive test that allows frequent longitudinal monitoring of cell-surface complement activity during treatment would provide novel information about the pharmacodynamic effect of therapy at the local level of specific organs, which represents a significant advancement to current methods limited to measurements of fluid-phase complement activity.

### SUMMARY

Provided herein are methods of using extracellular vesicles as a non-invasive, sensitive, and specific test to diagnose and/or monitor treatment response in patients with various complement-mediated diseases. In an aspect, the EVs can be considered as a liquid biopsy source, isolated from biological fluids including urine. The present disclosure is partly based on the discovery of complement deposition in EVs, which finding can be leveraged for many applications such as, ex-vivo analysis of patient samples as a surrogate for monitoring in vivo complement activity; methods for monitoring efficacy of drugs that modulate complement pathways; methods for monitoring patients over time; and methods for screening test compounds that modulate systemic and/or local tissue surface complement activity.

The present assay utilizes a bead-based immunocapture protocol with immunofluorescent detection to quantitate complement activity and dysregulation at the level of organ-specific tissue and modulation during treatment. Based on the exemplified proof-of-concept using urine samples, the present methods can be broadly applied to monitor any organ or tissue under complement attack in any liquid matrix using protein and tissue-specific detecting reagents including antibody-tagged beads and fluorophores. The principles of this assay and proof of concept performed in urine can potentially be expanded to blood and CSF for the analysis of shed EVs from other tissues/organs including EVs from tissue damaged by terminal complement complex deposition.

In an aspect, the disclosure provides an easy-to-use method to isolate and enrich for EVs and for semi-quantitative monitoring of complement on the surface of EVs before, during, and after therapeutic intervention. The present methods can be easily multiplexed and adapted to a wide variety of assay formats and/or combined with various analytical techniques, e.g., nanoparticle tracking analysis (NTA), mass spectroscopy (MS) or super resolution microscopy.

The present disclosure relates to the following non-limiting aspects:

The present invention provides a method of detecting complement activity in a biological sample, from a subject, comprising:
(a) isolating a portion of the biological sample comprising extracellular vesicles (EV) or a membrane-bound portion thereof with at least one first capture antibody or an antigen-binding fragment thereof to capture at least one first marker on the EV or the membrane-bound portion thereof, wherein the first marker comprises an EV-specific marker or a tissue-specific marker displayed on the EV,
(b) optionally contacting a portion of the sample with at least one second capture antibody or an antigen-binding fragment thereof to capture at least one second marker on the EV or the membrane-bound portion thereof and
(c) detecting, qualitatively or quantitatively, a presence or a level of a complement system-associated component on the captured EV or the membrane-bound portion thereof with at least one detection antibody or antigen-binding fragment thereof which is specific for the complement system-associated component, thereby detecting complement activation in the biological sample.

The first and/or second markers may, independently, be on the membrane of EVs if transmembrane (such as the membrane attack complex, or MAC), or inside EVs if soluble (such as C5); thus, “on” also includes “in” or “inside”.

In an embodiment, the first capture marker comprises an EV-specific marker and the optional second capture marker comprises a tissue-specific marker displayed on the EVs or a membrane-bound portion thereof. In an embodiment, both the first capture marker and the second capture marker are present, the first capture marker comprises an EV-specific marker and the second capture marker comprises a tissue-specific marker are detected.

In an embodiment, the extracellular vesicles in the biological samples are from a liquid biopsy such as urine. The biological sample is procured from a liquid biopsy protocol.

In an embodiment, the biological sample is from a tissue, an organ, or a body fluid. In an embodiment, the biological sample comprises EVs or membrane-bound portions thereof from bladder cells, kidney cells, whole blood, red blood cells, platelets, serum, plasma, a blood fraction other than serum or plasma, lymph, cerebrospinal fluid (CSF), saliva, tears, vaginal discharge, semen, glandular secretions, exudate, contents of cysts or feces, lavage, or ascites. In an embodiment, the biological sample comprises EVs or membrane-bound portions thereof from glomerular podocytes, convoluted tubule of the kidney, or bladder epithelium; or red blood cells (RBC). In an aspect, the biological sample is a urine sample. In an aspect, the biological sample is a red blood cells (RBC) sample.

In an embodiment, the first capture antibody or the antigen-binding fragment thereof is conjugated to a first solid support, optionally the second capture antibody or the antigen-binding fragment thereof is conjugated to a second solid support, and the detection antibody is conjugated to a detectable marker. In an embodiment, the method disclosed herein comprises contacting a portion of the biological sample with the first capture antibody or an antigen-binding fragment thereof and the second capture antibody or an antigen-binding fragment thereof, wherein the first capture antibody or the antigen-binding fragment thereof and the second capture antibody or the antigen-binding fragment thereof are conjugated to the same support or to different supports.

In an embodiment, the detectable marker is selected from the group consisting of: fluorophores, chromogens, and biotin. In an embodiment, the detectable marker is a fluorophore with an absorption maximum between about 500 nm and about 900 nm, between about 600 nm and about 1000 nm, or between about 500 nm and about 1000 nm, and an emission maximum between about 550 nm and about 900 nm, between about 600 nm and about 1000 nm, or between about 550 nm and about 1100 nm. In an embodiment, the detectable marker is phycoerythrin (PE) with or without conjugation to strepavidin. In an embodiment, the detectable marker is biotin for use with streptavidin-phycoerythrin (SAPE).

In an embodiment, the first and second solid supports are independently selected from the group consisting of: nanoparticles, microparticles, beads, magnetic beads, nanostructures, tissue culture plate, silica, and nanomatrices.

In an embodiment, the first marker is selected from the group consisting of extracellular vesicle-associated proteins; the optionally-contacted second marker is selected from the group consisting of tissue-specific extracellular vesicle-associated proteins; and the complement system-associated component is selected from the group consisting of: (a) components of the alternative complement pathway (AP), (b) components of the classical complement pathway (CP), and (c) components of the lectin complement pathway (MBL). In a preferred embodiment, the complement system-associated component is selected from the group consisting of (a) components of the alternative pathway (AP), and (b) components of the classical pathway (CP).

In an embodiment, the complement system-associated component is a protein selected from the group consisting of: C1q, C1r, C1s, C2, C2a, C2b, C3, C3a, C3b, iC3b, C4, C4a, C4b, C5, C5a, C5b, C6, C7, C8, C9, C5b-9 (Membrane Attack Complex, MAC), TF, CRP, pCRP, CD59, CD55, CR1, CR2, CR3, C5aR1, properdin, factor H, Factor H-related proteins and factor I. See FIG. 11 (Image adapted from Karasu, E., et al. Frontiers in Immunology 9(721), 2018).

In an embodiment, the first marker is selected from the group consisting of: ALIX, TSG101, CD9, CD63, CD81, CD40L, CD26, CD31, CD45, CD2, CD11a, CD24, CD55, CD59, CF106, CD56, CD51, CD82, Integrins, Tetraspanins, Annexins, HSP90, HSP70, Syntenin-1, ADAM10, EHD4, Actin, Rab5, clathrin, Flotillin-1, MHC I, MHC II, Actinin-4, GP96, EHD4, Mitofilin, and LAMP2; the second marker is selected from the group consisting of: podocalyxin (PODXL), aquaporin 2 (AQP 2), uroplakin1b (UPK1b), podocin (NPHS2), glycophorin A (GYPA), mucin-1, type 2 Na-K-2Cl co-transporter (NKCC2), aquaporin 1 (AQP 1), α-glutathione-S-transferase (alpha-GST), calbindin-D28K (CalD), megalin, cubilin, nephrin (Nphs1), Claudin-1, Annexin-V, synaptopodin (Synpo), Wilm's tumor protein (Wt1), Band 3, stomatin (STOM), BGP1, Globin, Glycophorin B, Rh polypeptides, and Rh glycoprotein; and the complement protein is selected from the group consisting of: C3, C5b-9, C4, C1q, and C9.

In an embodiment, the biological sample comprises EVs from the renal system and the second marker is a kidney-specific EV marker selected from the group consisting of podocalyxin (PODXL), aquaporin 2 (AQP 2), uroplakin1b (UPK1b) and podocin (NPHS2).

In an embodiment, the sample comprises EVs from red blood cells (RBC) and the second marker is an RBC-specific EV marker selected from glycophorin A (GYPA).

In an embodiment, the sample comprises EVs which are negative for CD81 as a first marker, negative for Uroplakin 1B (UPK1B) as a second marker, or negative for both CD81 as a first marker and UPK1B as a second marker.

In an embodiment, the capture and detection markers are present in the same EV or a membrane-bound portion thereof.

In an embodiment, the method further comprises determining if the subject suffers from a complement-mediated disease or is at risk of developing a complement-mediated disease, comprising comparing the presence or level of the component of the complement pathway on the EV or the membrane-bound portion thereof to a control. In an embodiment, the control comprises an identical sample from a healthy subject. In an embodiment, the method indicates that the subject suffers from a complement-mediated disease or is at risk of developing a complement-mediated disease if: the level or the presence of the component of the complement pathway on the EVs or the membrane-bound portion thereof obtained from the subject is enhanced as compared to the control, i.e., if the level obtained from the subject is greater compared to the sample from a control subject that is not diagnosed with a complement-mediated disease.

The present invention further provides a method for diagnosis or prognostic assessment of a complement-mediated disease in a subject, comprising:
(a) obtaining a sample comprising extracellular vesicles (EVs) or a membrane-bound portion thereof from the subject,
(b) contacting a portion of the sample with at least one first capture antibody or an antigen-binding fragment thereof to capture at least one first marker on the EVs or the membrane-bound portion(s) thereof;
(c) optionally contacting a portion of the sample with at least one second capture antibody or an antigen-binding fragment thereof to capture at least one second marker on the EVs or the membrane-bound portion(s) thereof;
(d) contacting the captured EVs or membrane-bound portions thereof comprising the at least one first marker and optionally the at least one second marker with at least one detection antibody or an antigen-binding fragment thereof specific for a complement system-associated component; and
(e) detecting, qualitatively or quantitatively, the detection antibody or the antigen-binding fragment thereof, to measure a presence of or a level of the component of the complement pathway on the EV or the membrane-bound portion thereof, wherein an elevation in the presence or level of the component of the complement pathway in the subject's sample compared to a control indicates that the subject has or is at risk of developing the complement-mediated disease.

The present invention also provides a method of indicating if a subject has or is at risk of having a complement-mediated disease comprising steps (a)-(e) discussed above.

In an embodiment, the first marker comprises an EV-specific marker or a tissue-specific marker displayed on EV. In an embodiment, the first marker comprises an EV-specific marker and the second marker comprises a tissue-specific marker displayed on EVs.

The present invention further provides a method for monitoring response to treatment of a complement-mediated disease with a complement modulator in a subject, comprising:
(a) obtaining a sample comprising extracellular vesicles (EV) or a membrane-bound portion thereof from the subject before and after the treatment,
(b) contacting a portion of the sample with at least one first capture antibody or an antigen-binding fragment thereof to capture at least one first marker on the EVs or the membrane-bound portion(s) thereof;
(c) optionally contacting a portion of the sample with at least one second capture antibody or an antigen-binding fragment thereof to capture at least one second marker on the EVs or the membrane-bound portion(s) thereof;
(d) contacting the captured EVs or membrane-bound portions thereof comprising the at least one first marker and optionally the at least one second marker with at least one detection antibody or an antigen-binding fragment thereof specific for a complement system-associated component; and
(e) detecting, qualitatively or quantitatively, the detection antibody or the antigen-binding fragment thereof, to measure a presence of or a level of the component of the complement pathway on the EV or the membrane-bound portion thereof, wherein an attenuation in the presence or level of the component of the complement pathway in the subject's sample after treatment with the complement modulator compared to before treatment with the complement modulator indicates that the subject is responding to the complement modulator.

In some embodiments, the complement modulator is a molecule listed in Table A. Preferably, the complement modulator is a molecule that modulates (e.g., increases or reduces; preferably reduces) the activity of a complement component selected from C1q, C1, C1s, C2, MASP-2, MASP-3, Factor D, Factor B, Properdin (Factor P), Factor H, C3/C5 Convertase, C5, C5a/C5aR, C3a/C3aR, C6, and/or CD59. Particularly, the complement modulator is a small molecule inhibitor of the complement component or an siRNA/RNAi targeting the complement component or an antibody which specifically binds to the complement component.

In an embodiment, the complement mediator is a complement 5 (C5) inhibitor, complement 5a (C5a) inhibitor, complement 5 receptor (C5R1) inhibitor, complement 3 (C3) inhibitor, Factor D (FD) inhibitor, Factor H (FH) inhibitor, Factor B (FB) inhibitor, MASP2 inhibitor, MASP3 inhibitor, properdin inhibitor, or a combination thereof.

In an embodiment, the disease is an inflammatory disease or a thrombotic disease. In an embodiment, the disease is a thrombotic hematological disease or a thrombotic nephrological disease. In an embodiment, the disease is a nephrological disease selected from the group consisting of atypical haemolytic uraemic syndrome (aHUS), C3 glomerulopathy (C3G), dense deposit disease (DDD), membranoproliferative glomerular nephritis (MPGN), lupus nephritis (LN), IgA nephropathy (IN), lupus nephritis (LN), membranous nephropathy (MN), complications due to hemodialysis in transplant patients, antibody-mediated rejection (AMR) and anti-neutrophil cytoplasmic antibody (ANCA)-associated vasculitis (AAV). In an embodiment, the disease is a hematological disease selected from the group consisting of paroxysmal nocturnal hemoglobinuria (PNH), atypical haemolytic uraemic syndrome (aHUS), secondary HUS due to solid organ transplant or hematopoietic stem cell transplants, Thrombotic microangiopathy (TMA), and cold agglutinin disease (CAD). In an embodiment, the disease is a neurological disease selected from neuromyelitis optica spectrum disorder (NMOSD), generalized myasthenia gravis (gMG), amyotrophic lateral sclerosis (ALS) and primary progressive multiple sclerosis (PPMS).

In an embodiment, the detection comprises immunoassay (e.g., ELISA or RIA), electron microscopy (EM), tandem mass tag (TMT), a luminescence assay (e.g., LUMINEX), or fluoroimmnoassay (FIA) (also called immunofluorescence assay (IF)). In an embodiment, the detection step is carried out in a multiplex format, i.e., wherein detection is carried out by measuring markers in several discrete tissues in one sample and/or monitoring multiple potential complement proteins & pathways in a single assay.

The present invention further provides a method of detecting complement activation in a subject's kidney tissue, comprising:
(a) contacting a urine sample from the subject, comprising extracellular vesicles (EV) or a membrane-bound portion thereof comprising a first marker which is an EV-specific marker or a tissue-specific marker displayed on EVs or membranes thereof, with a first capture antibody or an antigen-binding fragment thereof specific thereto, thereby capturing EVs or membranes containing the first marker;
(b) optionally contacting the urine sample with a second capture antibody or an antigen-binding fragment thereof to capture EVs or membrane-bound portion(s) thereof comprising the second capture marker which is different from the first marker; and
(c) detecting, qualitatively or quantitatively, a presence or a level of the component of the complement pathway on the captured EV or the membrane-bound portion thereof with an antibody or an antigen-binding fragment thereof specific to the component, thereby detecting complement activation in the urine sample;
wherein the EV-specific marker is selected from the group consisting of CD9, CD63, and CD81,
the tissue-specific marker is selected from the group consisting of:
   (1) podocalyxin (PODXL) specific for podocytes in the glomerulus;
   (2) aquaporin 2 (AQP2) specific for the convoluted tubule epithelium;
   (3) uroplakin1b (UPK1b) specific for bladder epithelium; and
   (4) glycophorin A (GYPA) specific for red blood cells (RBC), and the component of the complement pathway is selected from the group consisting of MAC, C3, C5b-9, C4, C1q, and C9.

In some embodiments, the disclosure relates to a method of screening a test compound for complement modulation comprising:
(a) obtaining a sample containing extracellular vesicles (EV) or a membrane-bound portion thereof from a subject suffering from a complement-mediated disease (e.g., an animal such as a mouse, rabbit, hamster, sheep, llama, dog, monkey, chimpanzee or human) before and after administration of the test compound to the subject;
(b) contacting a portion of the sample with at least one first capture antibody or an antigen-binding fragment thereof to capture at least one first marker on the EVs or the membrane-bound portion(s) thereof;
(c) optionally contacting a portion of the sample with at least one second capture antibody or an antigen-binding fragment thereof to capture at least one second marker on the EVs or the membrane-bound portion(s) thereof;
(d) contacting the captured EVs or membrane-bound portions thereof with at least one detection antibody or an antigen-binding fragment thereof specific for a complement system-associated component; and
(e) detecting, qualitatively or quantitatively, the detection antibody or the antigen-binding fragment thereof, to measure, a presence of or a level of the complement component on the EV or the membrane-bound portion thereof, wherein a modulation (e.g., increase or decrease; preferably decrease) in the presence or level of the complement component in the subject's sample after administration of the test compound compared to before administration of the test compound indicates that the test compound is capable of modulating complement.

In some embodiments, the test compound is specifically capable of modulating complement component selected from C1q, C1, C1s, C2, MASP-2, MASP-3, Factor D, Factor B, Properdin (Factor P), Factor H, C3/C5 Convertase, C5, C5a/C5aR, C3a/C3aR, C6, and/or CD59. In some embodiments, the test compound is a monoclonal antibody or small molecule or siRNA/RNAi. In some embodiments, the modulating activity of the test compound is compared to the activity of a molecule having complement modulating activity (e.g., positive control or standard), such as, a molecule provided in Table A.

The present invention furthermore provides for use of: at least one first capture antibody to capture at least one first target; at least one second capture antibody to capture at least one second target; and at least one detection antibody specific for a complement protein to detect an amount of the captured at least one first target, the captured at least one second target, or both.

Also provided are kits that contain one or more antibodies that bind to a biomarker as described herein. In some embodiments of the kits described herein, the kit is an immunoassay, e.g., enzyme-linked immunosorbent assay. Any of the kits described herein can be used to perform any of the methods described herein. In some embodiments, the kits can further include instructions for performing any of the methods described herein. Such kits can also include, as non-limiting examples, one or more of: reagents useful in preparing a sample, reagents useful for enriching extracellular vesicles, reagents useful for detecting binding of target proteins or component in a sample to immobilized antibodies, control samples that include purified target proteins/component, and/or instructions for use.

For example, kits useful in the methods described herein can include one or more (e.g., two, three, four, five, six, seven, eight, nine, ten, eleven, twelve, thirteen, fourteen, or fifteen) antibodies or fragments thereof that specifically bind to a biomarker as described herein. For example, the one or more antibodies provided in the kits can be immobilized on a surface (e.g., in the form of an ELISA assay or a gene-chip array).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1, panels (A) and (B), shows the relative abundance of EV markers in urine ExoQuick Enrichment by NTA. All Ab from Biolegend: Ms-α-CD9=PE: clone HI9a; Ms-α-CD63=PE: clone H5C6; Ms-α-CD81=PE: clone 5A6; Analysis done on ZetaView PMX110 by Particle Metrix GmbH.

FIG. 2 shows EM images of urine EV (A) and non-EV particles (B).

FIG. 3 shows the distribution of Feret Diameter in EM image objects.

FIG. 4 shows relative abundance of Top 25 proteins by PSM.

FIG. 5 shows detection of urine EV subsets by Luminex. CD9: clone MM2/57 (Southern Biotech); CD63: clone H5C6 (Biolegend); CD81: clone 1D6 (Abcam); Ms IgG: clone MG1-45 (Biolegend).

FIG. 6 shows that renal PODXL is detected only on CD9+ EV. (1): Rb-α-PODXL: USB cat #212672-biotin; (2): Rb-α-PODXL: LSBio cat #LS-C141161.

FIG. 7 shows that Luminex Beads can identify Glomerulus-specific EV. Rb-α-PODXL (USB 212672) can be detected on EV containing CD9 or CD40L but not on EV containing CD63 or CD81. Signal was found using 2 different α-PODXL antibodies. This can only occur if both proteins are located on the same structure.

FIG. 8 shows that Nephron-specific EV levels increase with disease. There are more PODXL+ EV in IgAN urine than in Control urine. Increases are seen in both CD9+/PODXL+ and CD40+/PODXL+ EV populations. CD63+ and CD81+ EV are still negative.

FIG. 9, panels (A) and (B), shows graphs showing Luminex Beads can measure complement on EV membranes. C3c and C5b-9 are detected in LN patient urine on both the CD9+ and PODXL+ beads but not on CD63+ or CD81+. These results have been confirmed in 10 LN, 6 IgAN, and 7 control samples (data not shown). Both LN and IgAN samples can have but are not limited to C3, C5b-9, C4 and C1q deposition on both PODXL+ and AQP2+ EV compared to control samples.

FIG. 10, panels (A), (B), and (C), shows graphs showing Glomerular C5b-9 deposition decreases with Ravulizumab treatment. aHUS patients may have any combination of C3, C5b-9, C1q and C4 (not shown) depositing on the podocyte membranes. C3 does not change during ravulizumab treatment. C5b-9 and C1q levels on EV are rapidly reduced during ravulizumab treatment.

FIG. 11 shows complement pathway-associated components.

FIG. 12 shows data from analysis of EV-enriched urine samples using a bead fluorescence-based assay method.

### DETAILED DESCRIPTION

### Definitions

The word “about” means a range of plus or minus 10% of that value, e.g., “about 5” means 4.5 to 5.5, unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5.

Where a range of values is provided in this disclosure, it is intended that each intervening value between the upper and lower limit of that range and any other stated or intervening value in that stated range is encompassed within the disclosure. For example, if a range of 1 mM to 8 mM is stated, it is intended that 2 mM, 3 mM, 4 mM, 5 mM, 6 mM, and 7 mM are also explicitly disclosed.

As used herein, the term “plurality” can be 2, 3, 4, 5, 6, 7, 8, 9, 10, or more.

As used herein, the term “detecting,” refers to the process of determining a value or set of values associated with a sample by measurement of one or more parameters in a sample and may further comprise comparing a test sample against reference sample. In accordance with the present disclosure, the detection of complement markers includes identification, assaying, measuring and/or quantifying one or more markers.

By the term “extracellular vesicle” is meant a lipid-based microparticle or nanoparticle, or protein-rich aggregate, present in a sample (e.g., a biological fluid) obtained from a subject. Extracellular vesicles are also referred to in the art and herein as exosomes, microvesicles or nanovesicles. In the present disclosure, an extracellular vesicle is between about 30 nm to about 1000 nm in diameter. Extracellular vesicles are secreted or shed from a variety of different mammalian cell types. Non-limiting examples of extracellular vesicles and methods for the enrichment of extracellular vesicles from a sample (e.g., a biological fluid) obtained from a mammalian subject are described herein. Additional examples of extracellular vesicles and methods for the enrichment of extracellular vesicles from a sample obtained from a mammalian subject are known in the art.

The term “membrane-bound” means any structure containing biological membranes, i.e., the outer coverings of cells and organelles that form a semi-permeable barrier. The term typically refers to structures containing phospholipids and proteins, derived from the outer cell membrane, or an organelle such as Golgi-apparatus, ER, the nucleus or mitochondria.

The term “membrane protein(s)” as used herein refers to proteins that interact with, or are part of, biological membranes of EVs. The membrane proteins may include, but are not limited to, integral membrane proteins and peripheral membrane proteins.

The term “disease specific membrane proteins” as used herein refers to membrane proteins that are associated with a specific disease, e.g., a complement-mediated disease such as aHUS. The disease specific membrane proteins may individually code for a disease, alternatively a group of disease specific membrane proteins may code for a disease.

By the term “sample” or “biological sample” is meant any biological fluid obtained from a mammalian subject (e.g., composition containing blood, plasma, serum or other blood fractions, lymph, urine, cerebrospinal fluid, ascites, saliva, breast milk, tears, vaginal discharge, amniotic fluid, lavage, semen, glandular secretions, exudate, contents of cysts and feces). In preferred embodiments, the sample comprises blood, serum, or plasma.

As used herein, the term “antibody” means an antibody, or a functional portion or fragment thereof, with a high binding affinity for an antigen, e.g., complement proteins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses natural, genetically engineered and/or otherwise modified antibodies of any class or subclass, including IgG and sub-classes thereof, IgM, IgE, IgA, and IgD.

The term “antigen” refers to any molecule, e.g., protein or a fragment thereof, that can specifically bind to an antibody or its antigen-binding fragment.

The term “antigen fragment” refers to a part of the antigen that can be recognized by the antigen-specific antibody.

“Beads” refer to particles whereupon desired capture antibodies have been immobilized. The beads generally are uniform in size within a single filtration matrix, but may vary in size ranging from about 1 nm to about 10,000 nm between different filtration matrices. The preferred shape is spherical; however, particles of any other shape can be employed since this parameter is immaterial to the nature of the invention.

“Strips” refer to elongated flat elements whereupon desired capture beads or desired capture antibodies have been immobilized. The strips generally are thin films uniform in size, but may vary in size and color depending on the amount and type of capture antibody immobilized.

The term “multiplex” refers to the detection of a plurality of marker across a single sample and/or detection of at least one marker across a plurality of samples.

The term “multiplex bead array platform” as used herein refers to any platform that utilizes particles or micro-particles that are distinguishable. Such distinguishable particles may be utilized, for example, to conduct multiplex immunoassays or molecular probe-based assays. A representative example includes Luminex® xMAP® Technology.

The term “classification dyes” as used herein refers to any mixture or combination of microparticles or beads used in the multiplex assay that have a mixture of classification dyes that enable the instrument to sort and classify the particles.

The term “reporter molecule” includes, without limitation, any and all fluorescent tags that are bound to the detection molecule in the assay. In the case of immunoassays designed to measure human antibody, the detection molecule can, for example, be goat anti-human IgG that is labeled with phycoerythrin.

A concise summary of the biologic activities associated with complement activation is provided, for example, in The Merck Manual, 16th Edition.

A “subject,” as used herein, can be any mammal. A subject can be, for example, a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. Included are, e.g., transgenic animals or genetically-altered (e.g., knock-out or knock-in) animals.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment, i.e., a particular therapeutic agent to treat a complement-mediated disease or disorder.

A “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9 and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H and I being negative regulators of the pathway. Components of the lectin pathway include, e.g., MBL2, MASP-1 and MASP-2. Complement components also include cell-bound receptors for soluble complement components. Such receptors include, e.g., C5a receptor (C5aR1 and C5aR2), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, or foreign structures found on microbial or artificial surfaces, etc. The term includes, without limitation, any complement regulator protein (e.g., Factor B, Factor D, Factor P, Factor H, Factor I, CD46, CD55, and CD59).

“Treating”, as used herein, refers to providing treatment, i.e., providing any type of medical or surgical management of a subject. The treatment can be provided to reverse, alleviate, inhibit the progression of, prevent or reduce the likelihood of a disorder or condition, or to reverse, alleviate, inhibit or prevent the progression of, prevent or reduce the likelihood of one or more symptoms or manifestations of a disorder or condition. “Prevent” refers to causing a disorder or condition, or symptom or manifestation of such not to occur for at least a period of time in at least some individuals. Treating can include administering a therapeutic agent/complement modulator to the subject following the development of one or more symptoms or manifestations indicative of a complement-mediated condition, e.g., to reverse, alleviate, reduce the severity of, and/or inhibit or prevent the progression of the condition and/or to reverse, alleviate, reduce the severity of, and/or inhibit or one or more symptoms or manifestations of the condition. According to the methods described herein, a composition/complement modulator can be administered to a subject who has developed a complement-mediated disease or condition or is at increased risk of developing such a disorder relative to a member of the general population. Such a composition/modulator can be administered prophylactically, i.e., before development of any symptom or manifestation of the condition. Typically in this case the subject will be at risk of developing the condition, for example, when exposed to a complement-activating composition, e.g., a particle or nanoparticle encapsulated therapeutic, e.g., a viral particle used in gene therapies or a therapeutic agent delivered by, for example, a lipid nanoparticle.

An “effective amount” of an active agent such as a therapeutic agent or complement modulator refers to the amount of the active agent sufficient to elicit a desired biological response (or, equivalently, to inhibit an undesired biological response). The absolute amount of a particular agent that is effective may vary depending on such factors as the desired biological endpoint, the agent to be delivered, the target tissue, etc. An “effective amount” may be administered in a single dose, or may be achieved by administration of multiple doses. An effective amount of the therapeutic agent, for example, may be an amount sufficient to relieve at least one symptom of a disorder. An effective amount may be an amount sufficient to slow the progression of a chronic and progressive disorder, e.g., to increase the time before one or more symptoms or signs of the disorder manifests itself or to increase the time before the individual suffering from the disorder reaches a certain level of impairment. An effective amount may be an amount sufficient to allow faster or greater recovery from an injury than would occur in the absence of the agent.

As used herein, the term “diagnosis” refers to methods by which a determination can be made as to whether a subject is likely to be suffering from a given disease or condition, including but not limited to complement-mediated diseases. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms, e.g., unexplained changes in vitals, or phenotypic, genotypic or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given characteristic.

The term “likelihood,” as used herein, generally refers to a probability, a relative probability, a presence or an absence, or a degree.

As used herein, the term “marker” refers to a characteristic that can be objectively measured as an indicator of normal biological processes, pathogenic processes or a pharmacological response to a therapeutic intervention, e.g., treatment with a complement inhibitor. Representative types of markers include, for example, molecular changes in the structure (e.g., sequence or length) or number of the marker, comprising, e.g., changes in level, concentration, activity, or properties of the marker.

The term “control,” as used herein, refers to a reference for a test sample, such as control EVs isolated from healthy cells, and the like. A “reference sample,” as used herein, refers to a sample of tissue or cells that may or may not have a disease that are used for comparisons. Thus a “reference” sample thereby provides a basis to which another sample, for example urine sample containing EVs, can be compared. In contrast, a “test sample” refers to a sample compared to a reference sample. The reference sample need not be disease free, such as when reference and test samples are obtained from the same patient separated by time.

The term “level” can refer to binary (e.g., absent/present), qualitative (e.g., absent/low/medium/high), or quantitative information (e.g., a value proportional to number, frequency, or concentration) indicating the presence of a particular molecular species.

The term “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance (e.g., +/−10%).

The term “complement-mediated” disorder or disease refers to a disorder in which its pathogenesis involves complement activation which exceeds a subject's self-protective mechanisms (e.g., self-protective proteins including CD 55 (decay accelerating factor), CD 59 (protectin), Factor H, and the like) and causes damage to the subject's cells and/or tissue.

As used herein, the term “at risk” for a disease or disorder refers to a subject (e.g., a human) that is predisposed to experiencing a particular disease. This predisposition may be genetic (e.g., or due to other factors (e.g., environmental conditions, hypertension, activity level, metabolic syndrome, etc.). Thus, it is not intended that the present disclosure be limited to any particular risk, nor is it intended that the present invention be limited to any particular type of disorder or dysfunction related to complement (e.g., aHUS).

Provided herein are methods for using extracellular vesicles from non-invasive liquid biopsy protocols as a non-invasive, sensitive and specific test to diagnose and/or monitor treatment response in patients with various complement-mediated diseases. A semi-quantitative method is described for monitoring the expression of surface complement on EV before, during, and after therapeutic intervention, using immunoprecipitation/immunoanalysis to isolate and analyze EV surface markers. These methods can successfully leverage EVs for ex-vivo monitoring of complement deposition as a surrogate for in vivo activity. Thus, with these methods researchers and physicians have a tool to directly monitor complement attack of discrete, identifiable tissues, such as regions of the kidney, throughout the course of treatment.

Biopsy is the current standard for the differential diagnosis of a subset of complement-mediated diseases. However, risk for serious complications limits patient selection and testing frequency. Extracellular vesicles offer an easily accessible window to monitor ongoing complement deposition on specific tissues, before and during treatment. Extracellular vesicles can also provide precise cellular identification of complement attack of the tissue. Any cell type or tissue of interest with a unique PM marker could be used to interrogate complement deposition.

### Immunoprecipitation/Immunoanalysis

The immunoprecipitation/immunoanalysis of the present disclosure uses a bead-based immunocapture protocol with immunofluorescent detection, which can allow the specific tissues under complement attack to be pinpointed and treatment response can be monitored. The technique can be broadly applied to monitor any organ or tissue under complement attack in any liquid matrix given the right set of antibody tools. One aspect of the methods discussed herein is a combination of EV enrichment and immunocapture of tissue-specific biomarkers present on the surface of shed EV. Capture biomarkers can be either canonical EV-specific (such as CD9, CD63, CD81) or tissue-specific proteins. Detection antibodies are specific for complement components, such as C5b-9, C3, C4, C1q, and C9. Examples of tissue-specific targets in urine include podocalyxin (PODXL) specific for podocytes in the glomerulus, aquaporin 2 (AQP2) specific for the convoluted tubule epithelium, or uroplakin1b (UPK1b) specific for bladder epithelium. A further aspect is that a positive signal can only occur when the capture and detection targets are present on the same structure.

For example, kidney biopsy is the current standard for the differential diagnosis of chronic kidney disease. However, it is an invasive procedure. Urinary extracellular vesicles (uEV), however, are a complex source of vesicles and biomarkers originating from every cell type along the renal system including all parts of the nephron. For example, PODXL is made only on podocytes in the glomerulus, AQP2 is derived from the proximal and distal convoluted tubules, and glycophorin A (GYPA) from RBCs can be used to gauge EV leak from the plasma into the filtrate. Certain disease states such as inflammation or malignancy increase the number of uEV shed by cells. Along with canonical EV markers, uEV carry plasma membrane-bound proteins from the parent cell which may provide novel insight into the “health” of the renal system.

In the Examples section and elsewhere, representative types of antibodies which are useful in carrying out various embodiments of the disclosure are provided, e.g., with information on the particular vendor and/or catalog number. It should be understood that the disclosure is not limited to the exemplary embodiments which utilize antibody detection regents from a particular vendor/manufacturer. Antibodies against the biomarkers/analytes of the disclosure can be obtained from any manufacturer, including, Biolegend (San Diego, CA), Southern Biotech (Birmingham, AL), United States Biological (USB; Salem, MA), Lifespan Biosciences (LSBIO; Seattle, WA), Abcam (Cambridge, United Kingdom), Cell Signaling Technology (Danvers, MA), and Sigma-Aldrich (St. Louis, MO). For example, rabbit anti-PODXL antibody can be purchased form USB (Catalog #212672), LSBIO (Catalog #LS-C141161), Abcam (Catalog #ab205350) and Sigma-Aldrich (Catalog #HPA002110); anti-CD9 antibody, clone MM2/57 can be purchased from Southern Biotech (Catalog #9310), EMD Millipore (Catalog #CBL162), VWR (Catalog #89366), and BIO RAD (Catalog #MCA469G). Antibodies may also be generated using conventional techniques, e.g., immunization of a mammal such as a mouse or rabbit and/or hybridoma technology.

### EV Characterization

Extracellular vesicles (EV), such as urinary extracellular vesicles (uEV) can be characterized by simultaneous immunoprecipitation/immunoanalysis, i.e., the Luminex® xMAP® Technology platform, and interrogated to determine surface phenotype and tissue origin of EV subsets.

Luminex Corporation® makes equipment and xMAP™ technology that combines immunoprecipitation with multiplex immunoassay. xMAP™ is a series of proprietary, color-coded microspheres that can be coated with capture antibody. Open-architecture xMAP™ technology enables multiplexing of biological tests (assays), reducing time, labor, and costs over traditional methods such as ELISA, western blotting, PCR, and traditional arrays. Systems using xMAP™ technology perform discrete assays on the surface of color-coded beads known as microspheres, which are then read in a compact analyzer. Using multiple lasers or LEDs and high-speed digital-signal processors, the analyzer reads multiplex assay results by reporting the reactions occurring on each individual microsphere.

Using the Luminex platform allows EV enrichment by immunoprecipitation prior to immunoassay. This removes the need for initial sample processing/EV enrichment, thus limiting potential false results.

Using a multiplex format allows monitoring of several discrete tissues in one sample. Working in a multiwell plate format allows monitoring of multiple potential complement proteins and pathways in one assay.

Generally, a capture bead is conjugated to a target-specific antibody. The conjugated bead is used to immunoprecipitate the target protein from the matrix (e.g., from a liquid sample, such as urine). A detection antibody conjugated to a label, such as phycoerythrin (PE) or to biotin+streptavidin-phycoerythrin (SAPE), is used to detect and quantitate the bead-captured target during analysis.

An array of xMAP beads coated with antibodies against canonical vesicle markers, such as CD9, CD63, and CD81, are used to enrich for discrete EV subsets from a biological sample, such as urine. Each bead set is then analyzed for the presence of other biomarkers that define and connect the subset phenotype to the tissue origin, and then analyzed further for the presence of complement-pathway proteins. In this way, a non-invasive “liquid biopsy” method is built to sample and monitor key biomarkers of specific tissue (such as renal) diseases for differential diagnosis, prognostic assessment and/or longitudinal monitoring of response to treatment.

### Target Protein Binding

Binding of target proteins to antibodies in solution or immobilized on an array can be detected using detection techniques known in the art. Examples of such techniques include immunological techniques such as competitive binding assays and sandwich assays; fluorescence detection using instruments such as confocal scanners, confocal microscopes, or CCD-based systems, and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces can be measured; techniques using radioisotopes, including conventional radioisotope binding and scintillation proximity assays (SPA); mass spectroscopy, such as liquid chromatography-mass spectrometry (LC-MS), HPLC-MS, matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as atomic force microscopy (AFM) and scanning electron microscopy (SEM); and techniques such as electrochemical, impedance, acoustic, microwave, and infrared (IR)/Raman detection.

### Measuring Complement Inhibition/Modulation

Any suitable method can be used for assessing the ability of an agent or composition containing the agent to inhibit complement activation (or any other relevant properties). A number of in vitro assays can be used. The ability of an agent to inhibit the classical or alternative complement pathway, for example, can be assessed by measuring complement-mediated hemolysis of erythrocytes (e.g., antibody-sensitized or unsensitized rabbit or sheep erythrocytes), by human serum or a set of complement components in the presence or absence of the agent. The ability of an agent to bind to one or more complement components such as C3, C5, C6, C7, C8, C9, factor B or factor D can be assessed using, for example, isothermal titration calorimetry or other methods suitable for performing in liquid phase. The ability of an agent to bind to a complement component can be measured, for example, using an ELISA assay. Other methods of use include surface plasmon resonance, equilibrium dialysis, etc.

Methods for measuring systemic or local complement activation taking place in vitro or in vivo and for determining the ability of a complement inhibitor to inhibit such activation are known in the art. Measurement of complement activation products such as C3a, C5a, C3bBb, C5b-9, etc., for example, provides an indication of the extent of complement activation. A decrease in the amount of such products indicates inhibition of complement activation. In some embodiments a ratio between an active cleavage product and its inactive desarginine (desArg) form is measured (e.g., C3a/C3adesArg). One of skill in the art can distinguish between classical, alternative, and lectin pathway activation by appropriate selection of the complement activation product(s) measured and/or appropriate activators of complement such as zymosan, lipopolysaccharide, immune complexes, etc. Other methods involve measuring complement-mediated hemolysis of red blood cells as a result of terminal complex formation.

Complement activation in vivo and/or its inhibition by a complement inhibitor, can be measured in an appropriate biological sample. Systemic complement activation and/or its inhibition by a complement inhibitor, can be measured in a blood sample, for example. Serial measurements beginning before administration of a complement inhibitor provide an indication of the extent to which the complement inhibitor inhibits complement activation and the time course and duration of the inhibition. It will be appreciated that a decrease in activation products may only become apparent once activation products present prior to administration of the complement inhibitor have been degraded or cleared.

In some embodiments, the complement modulator described herein can be formulated with additional active agents useful for treating or preventing a complement-associated disorder in a subject. Additional agents for treating a complement-associated disorder in a subject include, without limitation, an antihypertensive (e.g., an angiotensin-converting enzyme inhibitor), an anticoagulant, a corticosteroid (e.g., prednisone), or an immunosuppressive agent (e.g., vincristine or cyclosporine A); anticoagulants (e.g., warfarin (Coumadin), heparin, phenindione, fondaparinux, idraparinux); thrombin inhibitors (e.g., argatroban, lepirudin, bivalirudin, or dabigatran); fibrinolytic agent (e.g., ancrod, ε-aminocaproic acid, antiplasmin-a₁, prostacyclin, and defibrotide); a lipid-lowering agent; or an anti-CD20 agent such as rituximab.

### Comparative Methods

In some embodiments of the methods described herein, the methods include comparing a detected level of a complement biomarker to a reference level. In some embodiments, the reference represents levels of the biomarkers in a healthy control, i.e., a subject who has not been diagnosed with a complement-mediated disease. In some embodiments, the reference level is a median or cutoff level in a reference cohort, e.g., a cutoff defining a statistically significantly distinct group, e.g., a top or bottom tercile, quartile, quintile, or other percentile of the reference cohort.

Depending on the identity of the protein biomarker detected, levels above or below the reference level may be indicative of the presence of disease or increased risk, i.e., increased levels (i.e., levels above the reference) or decreased levels (i.e., levels below the reference) indicate the presence of disease or decreased.

In some embodiments, increased levels above a reference level are statistically significant, or are increased by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or 1000%. An increase, as described herein, can be determined by comparison to a threshold or baseline value (e.g., a threshold detection level of an assay for determining the presence or absence of a protein, or a reference level of protein in a reference subject (e.g., healthy reference). In some embodiments, decreased levels below a reference level are statistically significant, or decreased by at least 5%, 10%, 15%, 20%, 25%, 30%, 35%, 40%, 45%, 55%, 60%, 65%, 70%, 75%, 80%, 85%, 90%, or 95%. A decrease, as described herein, can be determined by comparison to a threshold or baseline value (e.g., a threshold detection level of an assay for determining the presence or absence of a protein, or a level of protein in a reference subject (e.g., a healthy reference subject or a subject who does not have a complement-mediated disease).

In some embodiments, the methods include calculating a ratio of the level of the protein biomarker in the subject sample to a reference level, and if the ratio is greater than a threshold ratio, determining that the subject has or is at risk of developing a complement-mediated disease as described herein. In some embodiments, whether the ratio is positive or negative is determined, and the presence of a positive or negative ratio indicates that the subject has or is at risk of developing a complement-mediated disease as described herein. Again, whether a positive or negative ratio indicates the presence of disease, or increased or decreased risk, can be readily determined according to the disclosures herein.

The complement system is comprised of several small proteins organized into a biochemical cascade serving to assist the immune system in the clearance of pathogens. The complement proteins circulate in the blood as inactive precursors. When stimulated by one of several triggers, proteases in the system cleave specific proteins to release cytokines and initiate an amplifying cascade of further cleavages.

### Measuring Complement Activation

Methods for measuring systemic or local complement activation taking place in vitro or in vivo are known in the art. Measurement of complement activation products such as C3a, C5a, C3bBb, C5b-9, etc., for example, provides an indication of the extent of complement activation. A decrease in the amount of such products indicates inhibition of complement activation. In some embodiments a ratio between an active cleavage product and its inactive desArg form is measured (e.g., C3a/C3a desArg). One of skill in the art can distinguish between classical, alternative, and lectin pathway activation by appropriate selection of the complement activation product(s) measured and/or appropriate activators of complement such as zymosan, lipopolysaccharide, immune complexes, etc. Other methods involve measuring complement-mediated hemolysis of red blood cells as a result of terminal complex (MAC) formation.

A number of different animal models with pathological features that resemble one or more features of a complement-mediated response are known in the art. An application of a complement modulator for treatment of complement-mediated diseases can be administered in various doses to mice, rats, dogs, primates, etc., that spontaneously exhibit a disorder or in which a disorder has been experimentally induced by subjecting the animal to a suitable protocol. The ability of the modulator to prevent or treat one or more signs or symptoms of the disorder is assessed using standard methods and criteria.

Compounds or complement modulators that show promising results in animal studies, such as acceptable safety and feasibility of administering a dose expected to effectively treat complement-mediated diseases in the relevant extravascular location in a human subject, may be tested in humans, e.g., using standard protocols and endpoints for clinical trials for therapies for the particular disorder under study.

The above-described compositions are useful in, inter alia, methods for treating or preventing a variety of complement-associated disorders in a subject. The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject (U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety). The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, a therapeutic agent is delivered to a subject by way of local administration. As used herein, “local administration” or “local delivery,” refers to delivery that does not rely upon transport of the composition or agent to its intended target tissue or site via the vascular system. The composition can be delivered, for example, by injection or implantation of the composition or agent or by injection or implantation of a device containing the composition or agent. Following local administration in the vicinity of a target tissue or site, the composition or agent, or one or more components thereof, may diffuse to the intended target tissue or site.

As outlined in detail in the Examples section, the assay methods of the present disclosure include measurement of changes in expression or levels of complement components in EVs. The EVs can be sourced from any biological sample, such as, urine, blood, lymph, CSF, ascites, pus, pleural fluid, hemoglobin, milk, amniotic fluid, synovial fluid, mucus, saliva, phlegm, aqueous humor, vitreous body, or the like.

In some embodiments, approaches such as differential ultracentrifugation, density gradient ultracentrifugation, size exclusion chromatography, ultrafiltration, and affinity/immunoaffinity capture methods may be used to enrich EVs from biological samples, although this step is optional. Preferably, the EV enrichment step is carried out before the first marker and optionally the second marker is contacted with the respective antibody or the antigen-binding fragment.

Next, EVs are characterized at the population level or single particle level. Here, the composition and levels of molecules in EVs, such as protein, lipid or nucleic acids, are analyzed. Techniques range from light-scattering microscopy or spectroscopy to molecular fingerprinting using proteomics. Overall levels of unique molecules can also be measured in the population. For single-particle analysis, specialized methods such as, optical microscopy and flow cytometry (for EVs >200 nm), single-particle interferometric reflectance imaging (>40 nm), nano-flow cytometry (˜40 nm), and electron microscopy, are used. Particularly, electron microscopy and flow cytometry permit study of individual EVs without extensive prior separation from a biological matrix.

In some embodiments, the EVs may be lysed using lysis buffers, e.g., RIPA buffer (20 mM Tris-HCl [pH 7.5], 150 mM NaCl, 1 mM Na₂ EDTA 1 mM EGTA, 1% NP-40, 1% sodium deoxycholate, 2.5 mM sodium pyrophosphate, 1 mM b-glycerophosphate, 1 mM Na₃ VO₄, 1 μg/ml leupeptin).

Characterization of EVs may include use of capture antibodies that specifically bind to markers (e.g., typically a protein or peptide, but could include other antigens) on the EVs. In some embodiment, a single capture antibody which is specific to an EV marker is used. In some embodiments, at least two capture antibodies are used, wherein a first capture antibody is specific to an EV-specific marker and a second capture antibody is specific to a tissue-specific marker displayed on the EVs.

EV specific markers include, without limitation, ALIX (UNIPROT: Q8WUM4), TSG101 (UNIPROT: Q99816), CD9 (UNIPROT: P21926), CD63 (UNIPROT: P08962), CD81 (UNIPROT: P60033), CD40L (UNIPROT: P29965), CD26 (UNIPROT: P27487), CD31 (UNIPROT: P16284), CD45 (UNIPROT: P08575), CD2 (UNIPROT: P06729; Q53F96), CD11a (UNIPROT: P20701), CD24 (UNIPROT: P25063), CD55 (UNIPROT: P08174), CD59 (UNIPROT: P13987; Q6FHM9), CF106 (UNIPROT: Q9H6K1), CD56 (UNIPROT: P13591), CD51 (UNIPROT: P06756), CD82 (UNIPROT: P27701), Integrins, Tetraspanins, Annexins, HSP90 (UNIPROT: P07900 (α1); Q14568 (α2); P14625 (OA HSP70 (e.g., UNIPROT: P11021), Syntenin-1 (UNIPROT: O00560), ADAM10 (UNIPROT: O14672), EHD4 (UNIPROT: Q9H223), Actin, Rab5 (UNIPROT: P20339 (α); P61020 (β); P51148 (γ)), clathrin (UNIPROT: P09496 (α); P09497 (β); Q00610 (H)), Flotillin-1 (UNIPROT: O75955), MHC I, MHC II, Actinin-4 (UNIPROT: O43707), GP96 (UNIPROT: P14625), EHD4 (UNIPROT: Q9H223), Mitofilin (UNIPROT: Q16891), and LAMP2 (UNIPROT: P13473), or a fragment thereof.

With respect to tissue specificity, EVs specific to glomerular podocytes, convoluted tubule of the kidney, or bladder epithelium are useful in the study of nephrological diseases and EVs from red blood cells (RBC) are useful in the study of hematological diseases. In some embodiments, the tissue specific EV includes, but is not limited to, the following:
PODXL (UNIPROT: O00592), expressed highly in glomerular podocytes, endothelial cells, glandular cells in fallopian tube, uterus and seminal vesicles;
AQP 2 (UNIPROT: P41181), found in the apical cell membranes of the kidney's collecting duct principal cells and in intracellular vesicles;
UPK1b (UNIPROT: O75841), found in asymmetrical unit membrane of bladder;
NPHS2 (UNIPROT: Q9NP85), expressed in the podocytes of fetal and mature kidney glomeruli;
GYPA (UNIPROT: P02724), major intrinsic membrane protein of the erythrocyte, specifically recognized by Mab TER119;
Mucin-1 (UNIPROT: P15941; Q7Z551), expressed on the apical surface of epithelial cells, especially of airway passages, breast and uterus; in T-cells and overexpressed in epithelial tumors, such as breast or ovarian cancer and also in non-epithelial tumor cells;
NKCC2 (UNIPROT: Q13621), kidney-specific renal Na, K & Cl cotransporter;
AQP1 (UNIPROT: P29972), expressed in plasma membranes of red cells and kidney proximal tubules;
GST-alpha such as GSTα1 (UNIPROT: P08263), predominantly expressed in small and large intestines and colon and weakly expressed in lymphocytes; GSTα2 (UNIPROT: P09210); GSTα3 (UNIPROT: Q16772); GSTα4 (UNIPROT: O15217), expressed at a high level in brain, placenta, and skeletal muscle; and GSTα5 (UNIPROT: Q7RTV2);
THP (UNIPROT: P07911), expressed in the tubular cells of the kidney, particularly by epithelial cells of the thick ascending limb of Henle's loop & distal convoluted tubule lumen;
Calbindin-D28K (CalB1; UNIPROT: P05937), found in the mammalian kidney; also expressed in a number of neuronal and endocrine cells, particularly in the cerebellum.
Megalin (UNIPROT: P98164), a multiligand binding receptor found in the plasma membrane of many absorptive epithelial cells;
Cubilin (CUBN; UNIPROT: O60494), expressed in the kidneys and small intestine;
Nephrin (Nphs1; UNIPROT: O60500), expressed in podocytes of kidney glomeruli;
Claudin-1 (CLDN1; UNIPROT: O95832), Strongly expressed in liver and kidney; expressed in heart, brain, spleen, lung and testis;
Annexin-V (ANXA5; UNIPROT: P08758), expressed in many tissues & blood cells;
Synaptopodin (Synpo; Q8N3V7), expressed in neurons and cerebral cortex;
Wilm's tumor protein (Wt1; P19544), expressed in the kidney and a subset of hematopoietic cells;
Band 3, anion transport protein (SLC4A1; P02730), expressed in erythrocytes (PMID: 7506871, PMID: 26542571); Isoform 2 is expressed in kidney (PMID: 7506871);
Stomatin (STOM; P27105), detected in erythrocytes and widely expressed;
Carcinoembryonic antigen-related cell adhesion molecule 1 (BGP1; P13688), expressed in columnar epithelial cells of the colon (PMID: 10436421), in T cells (PMID: 18424730), and expressed in granulocytes and lymphocytes;
Globin (e.g., Cytoglobin (CYGB); Q8WWM9), expressed in heart, stomach, bladder and small intestine;
Glycophorin B (GYPB; P06028), expressed on the renal endothelium and epithelium;
Rh polypeptides, Rh glycoprotein (RHAG; Q02094), expressed in erythrocytes.

Next, a presence or a level of a complement system-associated component on the captured EVs is detected. Any method may be used in the detection of the complement component, e.g., with a detection antibody or antigen-binding fragment thereof which is specific to the component; aptamers may also be used. Illustrative methods for detection include, e.g., immunohistochemical staining, western blotting, in cell western, immunofluorescent staining, ELISA, RIA, and fluorescent activating cell sorting (FACS), or any method known in the art.

There are generally two strategies used for detection of epitopes on antigens, direct methods and indirect methods. The direct method comprises a one-step staining, and may involve a labeled antibody (e.g., FITC conjugated antibody) reacting directly with the antigen on/in an EV. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, N Y, 1988), Harlow et al. (Using Antibodies, A Laboratory Manual, Cold Spring Harbor Laboratory, N Y, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech.

A wide variety of complement proteins may be detected using the present methods, including, e.g., (a) a component of the alternative pathway (AP), (b) a component associated with classical pathway (CP), and (c) a component associated with lectin pathway (MBL). In some embodiments, a plurality of components from different pathways, e.g., components from AP and CP, may be detected.

In preferred embodiments, the complement system-associated component is a component of AP or CP selected from, e.g., C3, C5b-9, C4, C1q, C9, C3b, iC3b, TF, CRP, pCRP, MAC, CD59, CF55, CR1, C5aR1, and C5; preferably MAC, C3, C5b-9, C4, C1q, and C9. A combination of the various components may be detected, e.g., a combination of C3 and C5. Wherein the component is a membrane attack complex (MAC), the method may include detection of any subunit or all subunits of MAC, e.g., C5b, C6, C7, C8 and C9 molecules.

In some embodiments, the methods of the disclosure include making measurements with EVs which lack certain markers, e.g., exosome-specific markers such as CD81 (UNIPROT: P60033), a protein expressed on B cells (PMID: 20237408), monocytes/macrophages (PMID: 12796480), hepatocytes (PMID: 12483205) and also CD4-positive T cells (PMID: 22307619).

In some embodiments, the methods of the disclosure include making measurements with EVs which are specific to tissues of non-interest, e.g., bladder in the context of urinary system. Examples include, e.g., UPK1B (UNIPROT: O75841), expressed in bladder epithelium.

Routine methods may be used to sort out EVs that are positive for non-desirable markers, e.g., FACS.

### Downstream Applications of the Assay Methods

The present methods for detecting complement activation in biological samples may be used in many downstream applications. For instance, the methods may be used to determine if a subject (from whom the biological sample is obtained) suffers from a complement-mediated disease or is at risk of developing a complement-mediated disease. The measurement of incidence or risk of complement-mediated disorder is carried out by comparing the presence or level of the component of the complement pathway on the EV or the membrane-bound portion thereof to a control (or reference standards). Typically, control or reference standard contains EVs isolated from an identical biological sample from a healthy subject. Routine methods may be carried out for processing and normalization of samples obtained from different subjects.

Likewise, the present methods may also be used to determine progression or regression of complement mediated diseases over time. This is carried out by comparing the presence or level of the component of the complement pathway on the EV or the membrane-bound portion thereof in the subject's sample at two different time points (e.g., t1 and t2, where t2>t1). A reduction in presence/level of complement at t2 compared to t1 indicates improved complement status and regression of complement disease; the reverse is true if presence/level of complement at t2 is increased compared to t1. The spacing between measurements (e.g., t2−t1) may depend on the nature of the disease and range from days to years, e.g., a few weeks, 1 month, 3 months, 6 months, 1 year, 2 years, 3 years, 5 years, 10 years, or more, e.g., 20 years.

### Measuring Efficacy of Drugs

The methods and assays of the instant disclosure can be used to monitor response to treatment of a complement-mediated disease with a complement modulator. A complement modulator is a molecule that can, directly or indirectly, modulate, e.g., activate or inhibit, a complement component, e.g., component protein. Without being limited in any manner, representative complement modulators whose efficacy may be tested according to the presently described methods are provided in Table A.

TABLE A
Therapeutic Approaches to Modulating the Complement System: Included below are
exemplary approaches to working up patients with complement-mediated diseases.

| Target | Therapeutic | Disease/Disorder pathology | Developer/Distributor |
|---|---|---|---|
| C1q | Anti-C1q mAb | Autoimmune disease (e.g., GBS, | Annexon |
| | | wAIHA, autoantibody disease) or | |
| | | neurodegenerative disease (e.g., | |
| | | ALS, HD, glaucoma/geographic | |
| | | atrophy (GA)) | |
| C1 | C1-INH (BERINERT, | hereditary angioedema (HAE) | CSL Behring, Salix |
| | RUCONEST, CYNRIZE) | | Pharma, Shire, |
| | | | respectively |
| C1s | Anti-C1s mAbBIVV020 or | α-C1s mAb for hemolytic events | True North Therapeutics |
| | activated anti-C1s Ab | in cold agglutinin disease (CAD) | Sanofi |
| | C1s peptide | CAD, warm antibody | Ra Pharma |
| | | autoimmune hemolytic anemia | |
| | | (wAIHA), neurodegenerative | |
| | | diseases (e.g., HD, AD, ALS, | |
| | | GBS). | |
| C2 | PRO-02 mAb | antibody-mediated inflammation | Prothix BV |
| | | and ischemia-reperfusion | |
| | | damage | |
| MASP-2 | Narsoplimab (α-MASP-2 | hematopoietic stem cell | Omeros |
| | mAb) | transplant associated thrombotic | |
| | | microangiopathy (HSCT-TMA); | |
| | | atypical hemolytic uremic | |
| | | syndrome (aHUS); IgA | |
| | | nephropathy (IgAN) | |
| MASP-3 | OMS906 (α-MASP-3 | paroxysmal nocturnal | Omeros |
| | mAb) | hemoglobinuria (PNH) and other | |
| | | complement-mediated diseases | |
| Factor D | Anti-FD mAb | GA/age-related macular | Genentech |
| | (Lampalizumab) | degeneration (AMD) | |
| | ACH-4471 or ACH-5228 | transfusion-dependent anemia; | Achillion |
| | | PNH w/ hemolysis (EVH) | |
| | BCX9930 | Complement-mediated disorders | Biocryst |
| | Various (see, Maibaum et | Various complement-mediated | Novartis |
| | al. (Nat Chem Biol. 2016 | diseases | |
| | 12(12): 1105-10)) | | |
| Factor B | Factor B siRNA IONIS- | IgA nephropathy (IgAN) and | Ionis |
| | FB-L_{RX} | other complement disorders | |
| | Anti-Factor B mAb | Various | Novelmed/Alexion |
| | LNP023 | PNH; C3-glomerulopathy (C3G); | Novartis |
| | | membranous glomerulonephritis | |
| | | and other kidney diseases | |
| Properdin | Anti-Properdin mAb | Renal diseases | Novelmed |
| | CLG561 Mab | AMD; GA | Novartis |
| Factor H | AMY-201, ‘mini-factor H’ | Periodontal disease | Amyndas |
| C3/C5 | Compstatin/Derivative— | GA; PNH; cold agglutinin disease | Apellis |
| Convertases | APL2, APL9 | (CAD); wAIHA; complement- | |
| | | dependent nephropathies (CDN); | |
| | | gene therapy by attenuating | |
| | | attack on AAV | |
| | Compstatin/Derivative— | C3G; PNH; periodontitis; kidney | Amyndas |
| | AMY-101 | transplantation | |
| | sCR1/TP10 | Transplant rejection | Celldex |
| | Mini-FH | PNH | Amyndas |
| | Mirococept | Ischemia reperfusion injury in | AdProTech |
| | | kidney allograft | |
| | CR2-Factor H/TT30 | PNH | Alexion |
| C5 | Anti-C5 mAb | PNH, aHUS, myasthenia gravis | Alexion |
| | | (gMG), neuromyelitis optica | |
| | | spectrum disorder (NMOSD) | |
| C5—Follow | Eculizumab biosimilar | See, above | Multiple (e.g., ABP 959 |
| up | mAbs | | (Amgen); Elizaria |
| | | | (Generium); SB12 |
| | | | (Samsung)) |
| | Nomacopan (Coversin; | PNH; aHUS; bullous pemphigoid | Akari |
| | rVA576) | (BP); uveitis; thrombotic | |
| | | microangiopathy (TMA); | |
| | | keratoconjunctivitis; rheumatoid | |
| | | arthritis (RA) | |
| | Zilucoplan (RA101495) | gMG; ALS; immune-mediated | RaPharma |
| | | necrotizing myopathy (IMNM); | |
| | | kidney diseases | |
| | Cemdisiran (ALN-CC5) | aHUS | Alnylam |
| | Zimura (ARC1905) | GA/AMD; neovascular AMD; | IVERIC/Opthotech A |
| | | Stargardt disease | |
| | Ravulizumab (ALXN | PNH; aHUS; gMG; NMOSD; | Alexion |
| | 1210) | Hematopoietic Stem Cell | |
| | | Transplant (HSCT)-TMA; ALS; | |
| | | complement-mediated TMA; | |
| | | severe COVID-19 | |
| | SOBI005 (affibody) | No disclosed indications | SOBI |
| | Tesidolumab (LFG316) | Transplant Associated | Novartis |
| | | Microangiopathy (TAM); | |
| | | Panuveitis; AMD; GA; PNH; | |
| | | kidney transplant | |
| | Pozelimab | PNH | Regeneron |
| | Crovalimab (SKY059) | PNH | Roche/Chugai |
| C5 | ALXN1720 | Various indications | Alexion |
| bispecific | | | |
| C5a/C5aR | Avacopan (CCX-168) | anti-neutrophil cytoplasmic | Chemocentryx |
| | | autoantibody (ANCA) vasculitis | |
| | Olendalizumab; | Various, e.g., GVHD | Alexion Pharmaceuticals |
| | ALXN1007 | | |
| | BDB-001 | COVID-19 | Staidson Biopharm/inflaRx |
| | IFX2 | chronic and autoimmune | inflaRx |
| | | inflammatory diseases | |
| C3a/C3aR | Various | Various | Both experimental and |
| | | | therapeutic |
| C6 | Anti-C6 mAb | MG; | Regenesance |
| | C6 Anti-sense RNA | Autoimmune diseases | |
| | CP010 | neurodegenerative disorders | Complement |
| | | | Pharma/Alexion |
| CD59 | Adeno associated vector | dry and wet forms of AMD | Hemera |
| | (AAV) CAGsCD59 | | |
| | (HMR59) | | |

By way of example, the disclosure relates to the following methods for monitoring efficacy of therapy of various complement-mediated diseases:

(A) A method for monitoring response to treatment of an autoimmune disease (e.g., GBS, wAIHA, autoantibody disease) or a neurodegenerative disease (e.g., ALS, HD, glaucoma/geographic atrophy (GA)) in a subject with an anti-C1q monoclonal antibody.

(B) A method for monitoring response to treatment of hereditary angioedema (HAE) in a subject with a C1-INH (e.g., BERINERT, RUCONEST, CYNRIZE).

(C)(1) A method for monitoring response to treatment of hemolytic events in cold agglutinin disease (CAD) in a subject with an anti-C1s monoclonal antibody (e.g., BIVV020 or activated anti-C1s antibody).

(C)(2) A method for monitoring response to treatment of a complement mediated disease selected from cold agglutinin disease (CAD), warm antibody autoimmune hemolytic anemia (wAIHA), neurodegenerative diseases (e.g., HD, AD, ALS, GBS) in a subject with a C1s peptide.

(D) A method for monitoring response to treatment of antibody-mediated inflammation or ischemia-reperfusion damage in a subject with an anti-C2 monoclonal antibody (e.g., PRO-02).

(E) A method for monitoring response to treatment of hematopoietic stem cell transplant associated thrombotic microangiopathy (HSCT-TMA); atypical hemolytic uremic syndrome (aHUS); or IgA nephropathy (IgAN) in a subject with an α-MASP-2 monoclonal antibody (e.g., Narsoplimab).

(F) A method for monitoring response to treatment of complement-mediated diseases such as paroxysmal nocturnal hemoglobinuria (PNH) in a subject with an α-MASP-3 monoclonal antibody (e.g., OMS906).

(G) A method for monitoring response to treatment of geographic atrophy (GA)/age-related macular degeneration (AMD) in a subject with an α-Factor D (FD) monoclonal antibody (e.g., lampalizumab).

(H) A method for monitoring response to treatment of transfusion-dependent anemia; PNH w/ hemolysis (EVH) in a subject with a small molecule Factor D (FD) inhibitor (e.g., danicopan (ACH-4471) or ACH-5228).

(I) A method for monitoring response to treatment of complement-mediated disorders in a subject with a small molecule Factor D (FD) inhibitor (e.g., BCX9930 or FD inhibitors in U.S. Pat. No. 9,388,199, which is incorporated by reference herein).

(J) A method for monitoring response to treatment of complement-mediated disorders such as IgA nephropathy (IgAN) in a subject with a Factor B (FB) inhibitor (e.g., Factor B siRNA IONIS-FB-L_{RX} or α-FB monoclonal antibody).

(K) A method for monitoring response to treatment of PNH; C3-glomerulopathy (C3G); membranous glomerulonephritis and other kidney diseases, in a subject with a Factor B (FB) inhibitor (LNP023).

(L) A method for monitoring response to treatment of renal diseases or degenerative disease (e.g., AMD or GA) in a subject with α-properdin (Factor P) monoclonal antibody (e.g., CLG561).

(M) A method for monitoring response to treatment of periodontal disease or PNH in a subject with a Factor H (FH) modulator (e.g., mini-factor H; AMY-201 or CR2-Factor H/TT30).

(N) A method for monitoring response to a subject's treatment of a complement-mediated disorder selected from GA, PNH, cold agglutinin disease (CAD), wAIHA, complement-dependent nephropathies (CDN), and C3G; or attenuation of periodontitis, transplantation rejection, ischemia reperfusion injury in allograft, or rejection of adeno-associated viral vector (AAV) in gene therapy, with a compstatin or a derivative thereof (e.g., APL2, APL9; AMY-101) or sCR1/TP10 or Mirococept.

(O) A method for monitoring response to a subject's treatment of PNH, aHUS, myasthenia gravis (gMG), neuromyelitis optica spectrum disorder (NMOSD) with an anti-C5 monoclonal antibody (e.g., eculizumab or a biosimilar thereof, e.g., ABP 959, Elizaria, or SB12).

(P)(1) A method for monitoring response to a subject's treatment of PNH; aHUS; bullous pemphigoid (BP); uveitis; thrombotic microangiopathy (TMA); keratoconjunctivitis; or rheumatoid arthritis (RA) with nomacopan (Coversin; rVA576).

(P)(2) A method for monitoring response to a subject's treatment of gMG; ALS; immune-mediated necrotizing myopathy (IMNM); or a kidney disease with Zilucoplan (RA101495).

(P)(3) A method for monitoring response to a subject's treatment of aHUS with an anti-C5 siRNA cemdisiran (ALN-CC5).

(P)(4) A method for monitoring response to a subject's treatment of GA/AMD; neovascular AMD; or Stargardt disease with Zimura (ARC1905).

(Q) A method for monitoring response to a subject's treatment of PNH; aHUS; gMG; NMOSD; hematopoietic stem cell transplant (HSCT)-TMA; ALS; complement-mediated TMA; or severe COVID-19, with an improved anti-C5 monoclonal antibody (e.g., ravulizumab).

(R)(1) A method for monitoring response to a subject's treatment of a complement-mediated disorder with an anti-C5 affibody (e.g., SOBI005).

(R)(2) A method for monitoring response to a subject's treatment of transplant associated microangiopathy (TAM); panuveitis; AMD; GA; PNH; or kidney transplant rejection with the anti-C5 antibody tesidolumab (LFG316).

(R)(3) A method for monitoring response to a subject's treatment of PNH with an anti-C5 antibody which is pozelimab or crovalimab (SKY059).

(S)(1) A method for monitoring response to a subject's treatment of anti-neutrophil cytoplasmic autoantibody (ANCA) vasculitis with Avacopan (CCX-168).

(S)(2) A method for monitoring response to a subject's treatment of GVHD or COVID-19 with an anti-C5 monoclonal antibody (e.g., olendalizumab (ALXN1007) or BDB-001 or IFX2).

(T)(1) A method for monitoring response to a subject's treatment of autoimmune diseases or myasthenia gravis (MG) with a complement C6 inhibitor selected from anti-C6 monoclonal antibody and C6 anti-sense RNA.

(T)(2) A method for monitoring response to a subject's treatment of neurodegenerative disorders with a complement C6 inhibitor CP010.

(U) A method for monitoring response to a subject's treatment of dry and wet forms of AMD with an adeno associated vector (AAV) encoding soluble CD59 (HMR59).

The above representative methods for measuring response to treatment is generally practiced in line with the above methods for detecting complement proteins in EVs, e.g., by (a) obtaining a sample containing extracellular vesicles (EV) or a membrane-bound portion thereof from the subject before and after the treatment, (b) contacting a portion of the sample with at least one first capture antibody or an antigen-binding fragment thereof to capture at least one first marker on the EVs or the membrane-bound portion(s) thereof; (c) optionally contacting a portion of the sample with at least one second capture antibody or an antigen-binding fragment thereof to capture at least one second marker on the EVs or the membrane-bound portion(s) thereof; (d) contacting the captured EVs or membrane-bound portions thereof with at least one detection antibody or an antigen-binding fragment thereof specific for a complement system-associated component; and (e) detecting, qualitatively or quantitatively, the detection antibody or the antigen-binding fragment thereof, to measure, a presence of or a level of the component of the complement pathway on the EV or the membrane-bound portion thereof, wherein a modulation (e.g., increase or decrease; preferably decrease) in the presence or level of the component of the complement pathway in the subject's sample after treatment with the complement modulator compared to before treatment with the complement modulator indicates the subject is responding to the complement modulator.

In some embodiments, the complement modulator is a modulator of C1q, C1, C1s, C2, MASP-2, MASP-3, Factor D, Factor B, Properdin (Factor P), Factor H, C3/C5 Convertase, C5, C5a/C5aR, C3a/C3aR, C6, or CD59; preferably a complement inhibitor such as monoclonal antibody or small molecule inhibitor or siRNA/RNAi, as shown in Table A.

The above methods are especially useful for testing the efficacy of molecules that inhibit terminal complement activation or activity, e.g., at the C5 axis or the C3 axis. Particularly, the above methods are especially applicable for testing the efficacy of C5 inhibitors, e.g., eculizumab or a follow-on molecule such as ravulizumab.

### Methods of Screening for Complement Modulating Test Compounds

In some embodiments, the disclosure relates to a method of screening a test compound for complement modulation comprising (a) obtaining a sample containing extracellular vesicles (EV) or a membrane-bound portion thereof from a subject suffering from a complement-mediated disease (e.g., an animal such as a mouse, rabbit, hamster, sheep, llama, dog, monkey, chimpanzee or human), wherein the sample is obtained before and after administration of the test compound; (b) contacting a portion of the sample with at least one first capture antibody or an antigen-binding fragment thereof to capture at least one first marker on the EVs or the membrane-bound portion(s) thereof, (c) optionally contacting a portion of the sample with at least one second capture antibody or an antigen-binding fragment thereof to capture at least one second marker on the EVs or the membrane-bound portion(s) thereof; (d) contacting the captured EVs or membrane-bound portions thereof with at least one detection antibody or an antigen-binding fragment thereof specific for a complement system-associated component; and (e) detecting, qualitatively or quantitatively, the detection antibody or the antigen-binding fragment thereof, to measure, a presence of or a level of the complement component on the EV or the membrane-bound portion thereof, wherein a modulation (e.g., increase or decrease; preferably decrease) in the presence or level of the complement component in the subject's sample after administration of the test compound compared to before administration of the test compound indicates that the test compound is capable of modulating complement. Preferably, the test compound is capable of modulating a complement which is C1q, C1, C1s, C2, MASP-2, MASP-3, Factor D, Factor B, Properdin (Factor P), Factor H, C3/C5 Convertase, C5, C5a/C5aR, C3a/C3aR, C6, or CD59, or a combination thereof.

In some embodiments, the modulating activity of the test compound is compared to the modulating activity of a molecule having complement modulating activity (e.g., positive control or standard), such as, a molecule provided in Table A.

Compounds that Inhibit C5 Activation or Activity

In representative embodiments, the complement modulator whose activity is tested or screened for in accordance with the above methods is a molecule that inhibits activation of C5, thereby reducing, suppressing and/or eliminating the complement-mediated effects (e.g., CSR or CARPA). Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

A complement inhibitor suitable for use in reducing, suppressing and/or eliminating the complement-mediated effects that occur during therapeutic administration of certain therapeutics (e.g., particle or nanoparticle encapsulated therapeutics) may bind to C5. Exemplary agents include antibodies, antibody fragments, polypeptides, small molecules, and aptamers. Exemplary antibodies are described in U.S. Pat. No. 6,534,058 and in Wang, et al., Proc. Natl. Acad. Sci. USA, 92:8955-8959, 1995. Exemplary compounds that bind to and inhibit C5 are described in U.S. Pat. Nos. 7,348,401 and 7,999,081. In certain embodiments the complement inhibitor is an antibody, small molecule, aptamer, or polypeptide that binds to substantially the same binding site on C5 as an antibody described in U.S. Pat. No. 6,534,058 or a peptide described in U.S. Pat. No. 7,348,401. U.S. Pat. No. 7,538,211 discloses aptamers that bind to and inhibit C5. RNAi agents that inhibit local expression of C5 or CSR can also be used in the methods described herein.

In other embodiments the agent is an antagonist of a C5a receptor (CSaR).

C5a is cleaved from the alpha chain of C5 by either alternative or classical C5 convertase. The cleavage site for convertase action is at, or immediately adjacent to, amino acid residue 733 of the alpha chain of C5a. A compound that would bind at, or adjacent to, this cleavage site would have the potential to block access of the C5 convertase enzymes to the cleavage site and thereby act as a complement inhibitor. A compound that binds to C5 at a site distal to the cleavage site could also have the potential to block C5 cleavage, for example, by way of steric hindrance-mediated inhibition of the interaction between C5 and the C5 convertase. Exemplary C5a receptor antagonists include a variety of small cyclic peptides such as those described in U.S. Pat. No. 6,821,950; U.S. Pub. No. 2009/0117171; and/or WO2006/099330, or the monoclonal antibody BB5.1 (Frei Y. et al., Mol. Cell. Probes, 1:141-9, 1987), the single chain variable fragment (scFV) of BB5.1, or the anti-BB5.1 Fab (Peng et al., J Clin Invest., 115(6):1590-1600, 2005), which prevent the formation of C5a and CSb.

In certain embodiments, the complement inhibitor comprises an anti-C5 antibody. Anti-C5 antibodies (or VH/VL domains derived therefrom) suitable for use herein can be identified using methods known in the art. Alternatively, art recognized anti-C5 antibodies can be used. Antibodies that compete with any of these art recognized antibodies for binding to C5 also can be used.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia, C. et al. (Nature, 342:877 83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat Chothia combined definition. In such embodiments, these regions can be referred to as “combined Kabat Chothia CDRs” (Thomas, T. et al., Mol. Immunol., 33:1389 401, 1996) exemplifies the identification of CDR boundaries according to Kabat and Chothia definitions.

Another exemplary anti-C5 antibody is antibody BNJ421, as described in WO2015/134894 and U.S. Pat. No. 9,079,949, the teachings of which are incorporated herein by reference.

The anti-C5 antibody can comprise, for example, a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

Another exemplary anti-C5 antibody is the 7086 antibody described in U.S. Pat. Nos. 8,241,628 and 8,883,158, the disclosures in which are incorporated by reference herein.

Another exemplary anti-C5 antibody is the 8110 antibody also described in U.S. Pat. Nos. 8,241,628 and 8,883,158, the disclosures in which are incorporated by reference herein.

Another exemplary anti-C5 antibody is the 305LO5 antibody described in U.S. Pat. No. 9,765,135, the disclosure in which is incorporated by reference herein.

Another exemplary anti-C5 antibody is the SKY59 antibody (Fukuzawa, T. et al., Sci. Rep., 7:1080, 2017, the disclosure in which is incorporated by reference herein).

Another exemplary anti-C5 antibody is the REGN3918 antibody (also known as H4H12166PP) described in US Pub. No. 2017/0355757 or WO2017218515, the disclosures in which are incorporated by reference herein.

In another embodiment, the antibody competes for binding with, and/or binds to the same epitope on C5 as, the above-mentioned antibodies (e.g., 7086 antibody, 8110 antibody, 305LO5 antibody, SKY59 antibody, or REGN3918 antibody). The anti-C5 antibody can have, for example, at least about 90% variable region amino acid sequence identity with the above-mentioned antibodies (e.g., at least about 90%, 91%, 92%, 93%, 94%, 95%, 96%, 97%, 98% or 99% variable region identity).

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can comprise, for example, one or more (e.g., two, three, four, five, six, seven, or eight or more) amino acid substitutions relative to the native human Fc constant region from which the variant human Fc constant region was derived. The substitutions, for example, can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples (WO2015134894 and U.S. Pat. No. 9,079,949 the disclosures of each of which are incorporated herein by reference in their entirety).

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281: 23514 24, 2006); (2) the M428L or T250Q/M428L substitutions (Hinton, P. et al., J. Biol. Chem., 279:6213 6, 2004; Hinton, P. et al., J. Immunol., 176:346 56, 2006); and (3) the N434A or T307/E380A/N434A substitutions (Petkova, S. et al., Int. Immunol., 18:1759 69, 2006). Additional substitution pairings, e.g., P257I/Q311I, P257I/N434H, and D376V/N434H, have also been described (Datta-Mannan, A. et al., J. Biol. Chem., 282:1709 17, 2007). The entire teachings of each of the cited references are hereby incorporated by reference.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three or two) amino acid substitutions, insertions or deletions relative to the native constant region from which it was derived. In some embodiments, the variant Fc constant region comprises one or more amino acid substitutions selected from the group consisting of: M252Y, S254T, T256E, N434S, M428L, V2591, T250I and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376 the disclosure of which is incorporated herein by reference in its entirety.

In some embodiments the precise location of these mutations may be shifted from the native human Fc constant region position due to antibody engineering. The 428L/434S double substitution when used in an IgG2/4 chimeric Fc, for example, may correspond to 429L and 435S as in the M429L and N435S variants described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan or tyrosine for threonine at position 250; phenylalanine, tryptophan or tyrosine for methionine at position 252; threonine for serine at position 254; glutamic acid for arginine at position 255; aspartic acid, glutamic acid or glutamine for threonine at position 256; alanine, glycine, isoleucine, leucine, methionine, asparagine, serine, threonine or valine for proline at position 257; histidine for glutamic acid at position 258; alanine for aspartic acid at position 265; phenylalanine for aspartic acid at position 270; alanine or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan or tyrosine for threonine at position 307; alanine, phenylalanine, isoleucine, leucine, methionine, proline, glutamine or threonine for valine at position 308; alanine, aspartic acid, glutamic acid, proline or arginine for leucine or valine at position 309; alanine, histidine or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan or tyrosine for methionine at position 428; lysine for histidine at position 433; alanine, phenylalanine, histidine, serine, tryptophan or tyrosine for asparagine at position 434; and histidine for tyrosine or phenylalanine at position 436, all in EU numbering.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3 or 0.2) nM.

In other embodiments, the [(K_{D} of the antibody for C5 at pH 6.0 at 25° C.)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

Compounds that Inhibit Factor B Activation or Activity

In certain embodiments the complement inhibitor inhibits activation of factor B. The complement inhibitor can bind to factor B, for example, thereby inhibiting activation. Exemplary agents include antibodies, antibody fragments, peptides, small molecules, and aptamers. Exemplary antibodies that inhibit factor B are described in U.S. Pat. Pub. No. 20050260198. In certain embodiments the isolated antibody or antigen-binding fragment selectively binds to factor B within the third short consensus repeat (SCR) domain. In certain embodiments the antibody prevents formation of a C3bBb complex. In certain embodiments the antibody or antigen-binding fragment prevents or inhibits cleavage of factor B by factor D. In certain embodiments the complement inhibitor is an antibody, small molecule, aptamer, or polypeptide that binds to substantially the same binding site on factor B as an antibody described in U.S. Pat. Pub. No. 20050260198, or is an RNAi agent that inhibits local expression of factor B. Peptides that bind to and inhibit factor B can be identified using methods known in the art.

Compounds that Inhibit Factor D Activity

In certain embodiments the complement inhibitor inhibits factor D. The complement inhibitor may bind to factor D, for example, thereby inhibiting factor D. Exemplary agents include antibodies, antibody fragments, peptides, small molecules, and aptamers. While factor D has been suggested as a desirable target for systemic complement inhibition as a result of its relatively low serum concentration and ability to inhibit alternative pathway activation, the present disclosure is directed to the therapeutic potential of locally administered agents that inhibit factor D. Exemplary antibodies that inhibit factor D are described in U.S. Pat. No. 7,112,327. In certain embodiments the complement inhibitor is an antibody, small molecule, aptamer, or polypeptide that binds to substantially the same binding site on factor D as an antibody described in U.S. Pat. No. 7,112,327. Exemplary polypeptides that inhibit alternative pathway activation and are believed to inhibit factor D are disclosed in U.S. Pub. No. 20040038869. Peptides that bind to and inhibit factor D can be identified using methods known in the art.

### Multimodal Complement Inhibitors/Modulators

The complement inhibitor useful in the methods described herein can bind to more than one complement protein and/or inhibit more than one step in a complement activation pathway. Such complement inhibitors are referred to herein as “multimodal.”

The complement inhibitor can be, for example, a virus complement control protein (VCCP) (U.S. Pat. No. 7,947,267 and WO2006042252). In certain embodiments the VCCP is a poxvirus complement control protein (PVCCP) or a herpesvirus complement control protein (HVCCP).

The VCCP may inhibit the classical complement pathway, the alternative complement pathway, the lectin pathway, or any two or more of these. The VCCP, e.g., a PVCCP, can bind to C3b, C4b, or both, for example. The PVCCP can comprise one or more putative heparin binding sites (K/R-X-K/R) and/or possesses an overall positive charge. In some embodiments, the PVCCP comprises at least 3 SCR modules (e.g., modules 1-3), e.g., 4 SCR modules. The PVCCP protein can be a precursor of a mature PVCCP (i.e., can include a signal sequence that is normally cleaved off when the protein is expressed in virus-infected cells) or can be a mature form (i.e., lacking the signal sequence).

Vaccinia complement control protein (VCP) has been shown to inhibit the classical pathway of complement activation via its ability to bind to C3 and C4 and act as a cofactor for factor I mediated cleavage of these components as well as promoting decay of existing convertase (Kotwal, G. et al., Science, 250:827-30, 1990; McKenzie, R. et al., J. Infect. Dis., 166:1245-50, 1992). It has also been shown to inhibit the alternative pathway by causing cleavage of C3b into iC3b and thereby preventing the formation of the alternative pathway C3 convertase (Sahu, A. et al., J. Immunol., 160, 5596-604, 1998). VCP thus blocks complement activation at multiple steps and reduces levels of the proinflammatory chemotactic factors C3a, C4a, and C5a. Homologs of VCPs, such as smallpox inhibitor of complement enzymes (SPICE) or any of the portions thereof that inhibit complement activation, e.g., SPICE-related polypeptides containing four SCRs, can be used in the methods described herein. Moreover, complement control proteins from cowpox virus (IMP) or monkeypox virus (MCP) can also be used in the methods described herein.

In addition to VCCPs, a number of other viral proteins exist that interfere with one or more steps in a complement pathway and can be used in the methods described herein, e.g., glycoprotein gC from HSV-1, HSV-2, VZV, PRV, BHV-1, EHV-1, and EHV-4 (Schreurs, C. et al., J. Virol., 62:2251-7, 1988). With the exception of VZV, the gC protein encoded by these viruses binds to C3b (Friedman, H. et al., Nature, 309:633-5, 1984) and gC1 (from HSV-1) accelerates decay of the classical pathway C3 convertase and inhibits binding of properdin and C5 to C3. The foregoing proteins are referred to collectively as virus complement interfering proteins (VCIPs). By any of a variety of means, such as interfering with one or more steps of complement activation, accelerating decay of a complement component, and/or enhancing activity of a complement regulatory protein, these VCIPs are said to inhibit complement. Any of these proteins, or derivatives thereof, e.g., fragments or variants thereof, can be used as a therapeutic agent in the methods described herein.

Additional Complement Inhibiting Agent. Modulators, Mixtures, and Modifications

A variety of other complement inhibitors can be used in various embodiments of the methods described herein. In some embodiments, the complement inhibitor is a naturally occurring mammalian complement regulatory protein or a fragment or derivative thereof. The complement regulatory protein can be, for example, CR1, DAF, MCP, CFH or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can be used. The compounds known as TP10 or TP20 (Avant Therapeutics), for example, can be used. C1 inhibitor (C1-INH) is also of use. In some embodiments a soluble complement control protein, e.g., CFH, is used. In some embodiments, the polypeptide is modified to increase its solubility.

Inhibitors of C1s are of use (e.g., U.S. Pat. No. 6,515,002 describes compounds (furanyl and thienyl amidines, heterocyclic amidines, and guanidines) that inhibit C1s; U.S. Pat. Nos. 6,515,002 and 7,138,530 describe heterocyclic amidines that inhibit C1s; U.S. Pat. No. 7,049,282 describes peptides that inhibit classical pathway activation; U.S. Pat. No. 7,041,796 discloses C3b/C4b Complement Receptor-like molecules and uses thereof to inhibit complement activation; U.S. Pat. No. 6,998,468 discloses anti-C2/C2a inhibitors of complement activation; U.S. Pat. No. 6,676,943 discloses human complement C3-degrading protein from Streptococcus pneumoniae).

Combination therapy using two or more complement inhibitors is encompassed in the methods described herein. The two or more complement inhibitors may be provided in the same composition. In certain embodiments the complement inhibitors bind to two or more different complement components. In certain embodiments the complement inhibitors bind to two or more different soluble complement proteins. In certain embodiments the complement inhibitors inhibit activation or activity of at least two complement proteins selected from C3, C5, C6, C7, C8, C9, factor B, and factor D.

### EXAMPLES

### Example 1

Characterization of urinary extracellular vesicles (uEV) by simultaneous immunoprecipitation/immunoanalysis using the Luminex® xMAP® Technology platform.

An array of xMAP beads coated with antibodies was used against canonical vesicle markers CD9, CD63, and CD81 to enrich for discrete EV subsets from urine. Each bead set was then analyzed for the presence of other biomarkers that define and connect the subset phenotype to the tissue origin. In this way, a non-invasive “liquid biopsy” method was built to sample and monitor key biomarkers of specific renal diseases for differential diagnosis, prognostic assessment and/or longitudinal monitoring of response to treatment.

Electron Microscopy:

Electron Microscopy Sample Preparation:
1) start with fresh urine+protease inhibitor. Centrifuge 2.5 K×g; collect supernatant;
2) enrich using ExoQuick Ultra TC kit to 300 uL;
3) NTA of product ±ExoGlow for EV and total particle density;
4) concentrate prep to 30 uL in Microcon 10K mwco;
5) add equal volume of Karnovsky's fixture;
6) repeat NTA for total particle density only;
7) process for imaging.

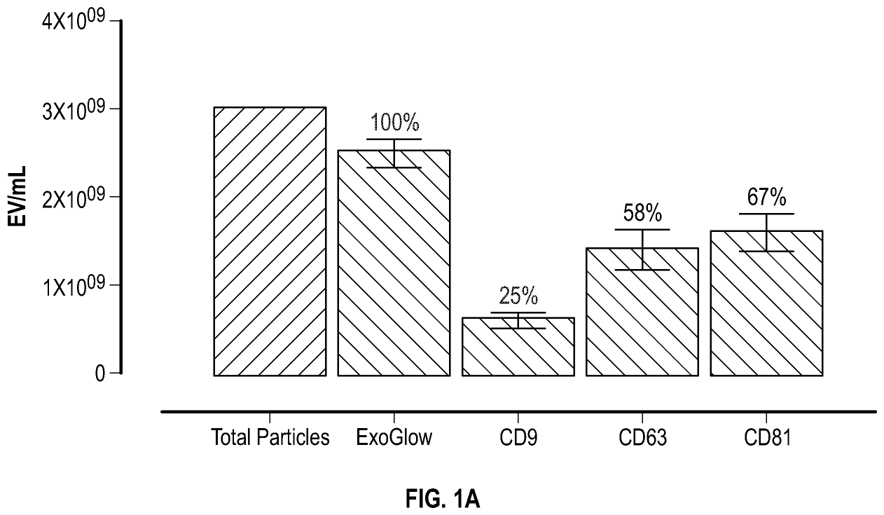

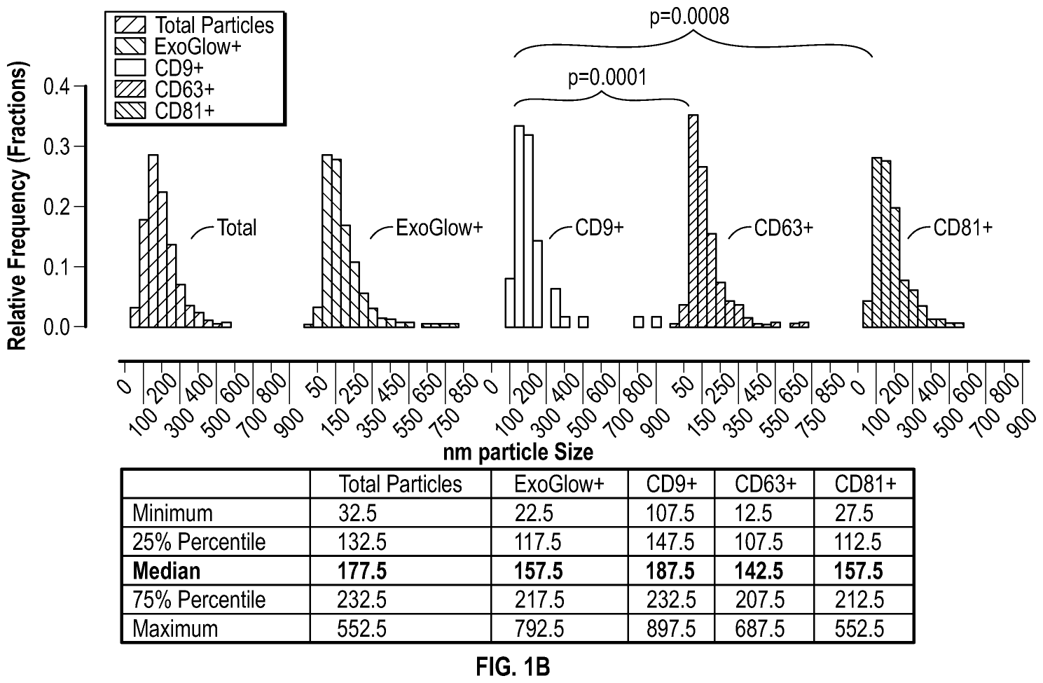

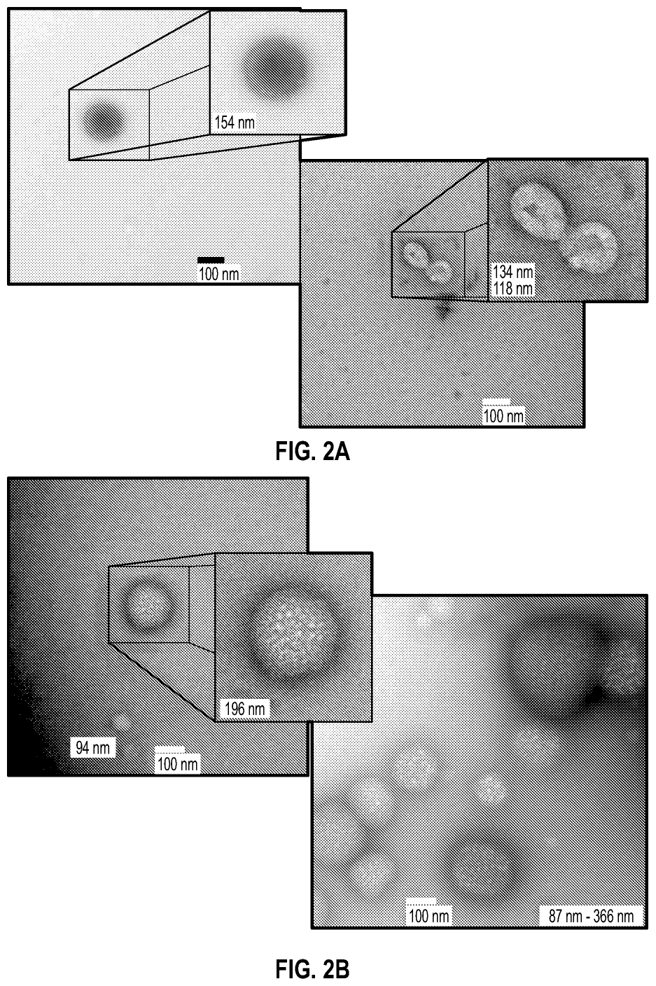

Relative abundance of EV markers in urine ExoQuick Enrichment by NTA is shown in FIG. 1. The EM imaging of urine EV and non-EV particles is shown in FIG. 2. Distribution of Feret diameter in EM image objects in shown in FIG. 3.

Mass Spectroscopy:

Tandem Mass Tag (TMT) Labeling:
1) start with fresh urine+protease inhibitor. Centrifuge 300× g; collect supernatant;
2) enrich using ExoQuick-Ultra TC kit;
3) reduce and amidate sulfhydryl bonds;
4) precipitate total proteins in 80% acetone, overnight, −20° C.;
5) reconstitute in buffer and digest with trypsin;
6) label fragments with TMT™ Isobaric label;
7) combine samples and dry down;
8) separate fragments by Ultimate3000;
9) analyze the LC fractions by OrbiTrap Lumos.

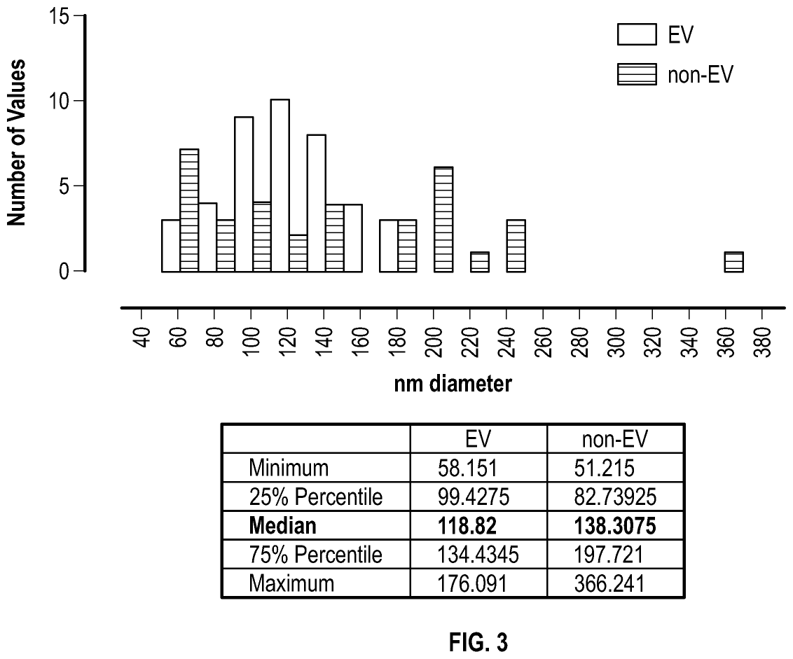

The relative abundance of Top25 proteins by PSM is shown on FIG. 4.

TABLE 1
mean abundance of key biomarkers by TMT

| | | | ExoQuick | |
|---|---|---|---|---|
| | | S300 | Urine | |
| | | Urine | (fold of S300) | Comments |
| Canonical | ALIX | 27.4 | 210.6 | Average of |
| EV Markers | | | (7.7) | 4 different |
| | | | | ALIX fragments |
| | CD63 | 7.2* | 281.7 | No CD81 |
| | | | (38.6) | detected |
| | CD9 | 9.8 | 311.7 | |
| | | | (31.8) | |
| | Tsg101 | 40.9 | 185.1 | |
| | | | (4.5) | |
| Nephron/ | AQP2 | 10.9 | 252.7 | |
| Renal | | | (23.2) | |
| Markers | NPHS2 | 73.5^{ł} | 248.8 | |
| | | | (3.4) | |
| | PKD-1 | Not found | | |
| | PODXL | 50.7 | 168.8 | |
| | | | (3.3) | |
| | UPK1b | Not found | | |
| Soluble | Cyst C | 19.1 | 76.5 | |
| Urine | | | (4.0) | |
| | UMOD | 49.5 | 314.8 | |
| | | | (6.4) | |
| Lipid | APOA1 | 68.1 | 33.3 | |
| Contaminant | | | (0.5) | |
| Markers | APOA4 | 46.2 | 27.7 | |
| | | | (0.6) | |
| | APOD | 75.7 | 38.9 | |
| | | | (0.5) | |
| | APOE | 40.7 | 37.9 | |
| | | | (0.9) | |

Luminex Assay Development:

dUC Sample Preparation:
1) start with fresh urine+protease inhibitor. Centrifuge 300× g; collect supernatant;
2) Transfer 5300 to Ultra-Clear™ tubes and centrifuge 200K× g overnight, 4° C.;
3) Resuspend and pool the pellets in cold PBS+protease inhibitor+200 mg/mL DTT;
4) Centrifuge 200K× g for 6 hr, 4° C.;
5) Resuspend the pellet in PBS+protease inhibitor;
6) Aliquot and store ≤−70° C.

FSL-Biotin Tagging of EV:

FSL-biotin is a Kode™ Technology construct designed to label hydrophobic surfaces with biotin. FSL-biotin is comprised of a monomer of biotin (vitamin B7) conjugated to a maleimide-bearing carboxymethylglycine based linker, in turn conjugated to an activated adipate derivative of dioleoylphosphatidylethanolamine (KODE Biotech website: kodebiotech.com/sales/products/product_info.php?id=129&cat=rdt (Accessed 27 Sep. 2018).

Luminex Assay Design:
1) Use 1E6 EV/well of dUC enriched uEV;
2) Add an equal volume of labeled MagPlex™ beads;
3) Incubate overnight, 4° C., gentle shaking;
4) Wash 2× with PBS;
5) Add either
   a. Biotinylated polyclonal antibody+SAPE in PBS/BSA
   b. FSLB+SAPE in PBS;
6) Incubate 1 hr, RT, gentle shaking;
7) Wash 2× with PBS;
8) Wash 2× with sheath fluid;
9). Read on Luminex machine, High PMT.

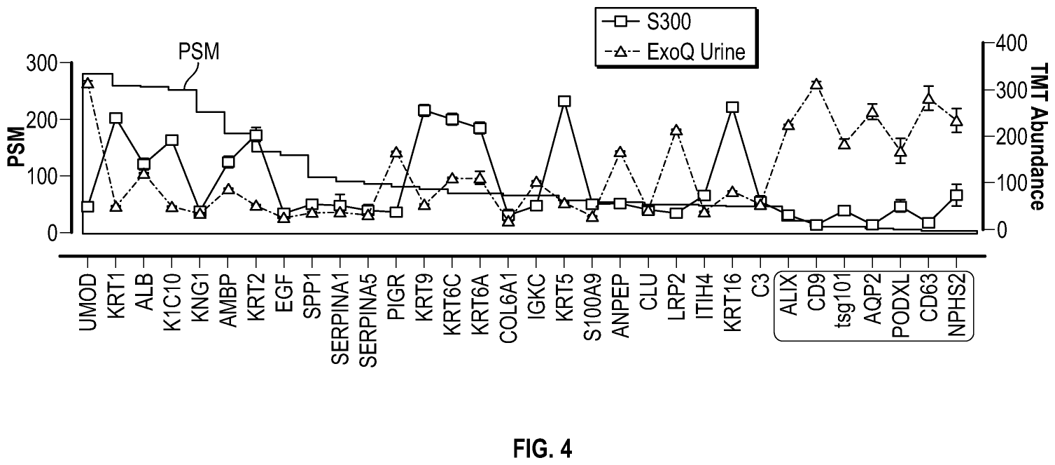

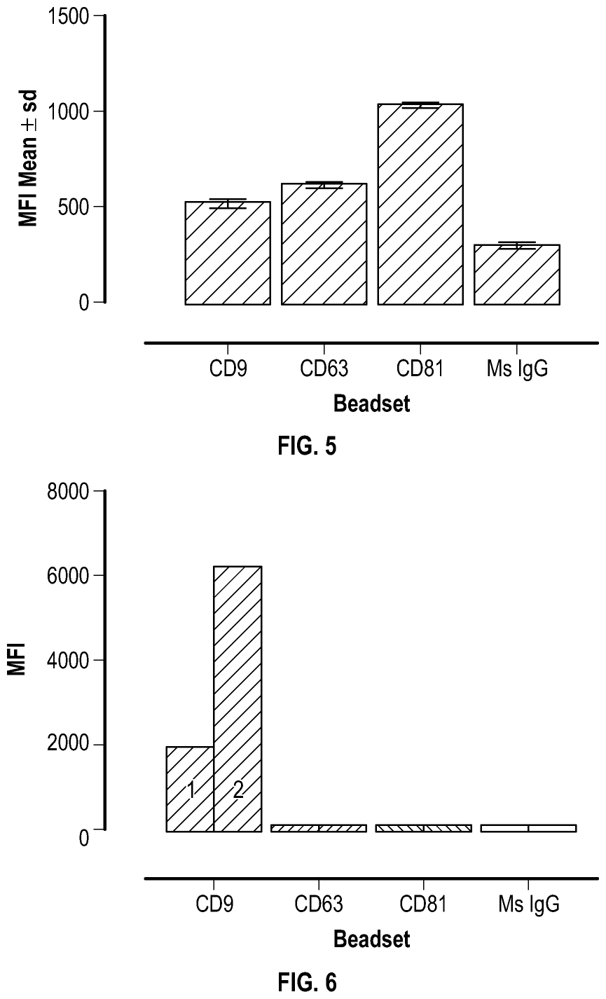

FIG. 5 shows detection of urine EV subsets by Luminex. FIG. 6 shows renal PODXL detected only on CD9⁺ EV.

These results indicate this protocol enriches for uEV & the CD9+ subset contains PODXL, a marker of renal tissue origin. Optimizing this protocol with improved sensitivity and specificity should improve uEV enrichment and identify more renal biomarkers. Once optimized, this methodology may be applied to various disease populations for differential analysis compared to healthy donors.

### Example 2

Assessment of complement deposition in organs and tissues by simultaneous immunoprecipitation/immunoanalysis of urinary extracellular vesicles (uEV) using the Luminex® xMAP® Technology platform.

An array of xMAP beads coated with antibodies was used against defined regions of the nephron: podocalyxin (PODXL) for the glomerular podocytes or aquaporin 2 (AQP2) for the convoluted tubules. Each bead set was then analyzed for the presence of complement markers that have been deposited on the plasma membrane (PM) of the cell of origin. In this way, urine EV can be used to monitor complement deposition in the nephron for differential diagnosis, prognostic assessment and/or longitudinal monitoring of response to treatment.

EV carry surface markers from their parent cell. These markers can be used to immunoisolate EV based on cell origin. PODXL is made only on podocytes in the glomerulus. AQP2 is derived from the proximal and distal convoluted tubules. Glycophorin A (GYPA) from RBCs can be used to gauge EV leak from the plasma into the filtrate. Circulating EV concentrations are elevated in inflammatory and thrombotic conditions. Some EV carry complement regulators on their surfaces such as CD55 and CD59. Cells can use EV to shed low concentrations of MAC complex from their surfaces. EV may act as a locus for thrombin generation.

Luminex Assay Development:

Assay Design Goals:

To be a useful and practical substitute for kidney biopsy, the present assay typically includes features such as (a) ability to start with frozen samples; (b) require minimal processing to achieve goals; (c) simplicity and the ability to analyze multiple samples simultaneously and consistently; and (e) retain membrane integrity. This means pre-enrichment selection of vesicle populations prior to luminex bead IP is typically minimized.

Luminex Assay Design:
1) Use 50 uL/well of urine diluted 1:2 in PBS/BSA;
2) Add 1000/well of labeled xMAP™ beads;
3) Incubate overnight, 4° C., gentle shaking;
4) Wash 2× with PBS;
5) Add biotinylated polyclonal antibody+SAPE in PBS/BSA;
6) Incubate 1 hr, RT, gentle shaking;
7) Wash 2× with PBS;
8) Wash 2× with sheath fluid;
9) Read on Luminex machine, High PMT.

TABLE 2
Beadsets Currently in Use

| Antibody Specificity | Function |
|---|---|
| 1. Mouse IgG | Negative Control |
| 2. CD9 | Canonical EV marker |
| 3. CD63 | Canonical EV marker |
| 4. CD81 | Canonical EV marker |
| 5. CD40L | Canonical large EV marker |
| 6. PODXL | Glomerulus marker |
| 7. AQP2 | Convoluted tubule marker |
| 8. GYPA | RBC marker |

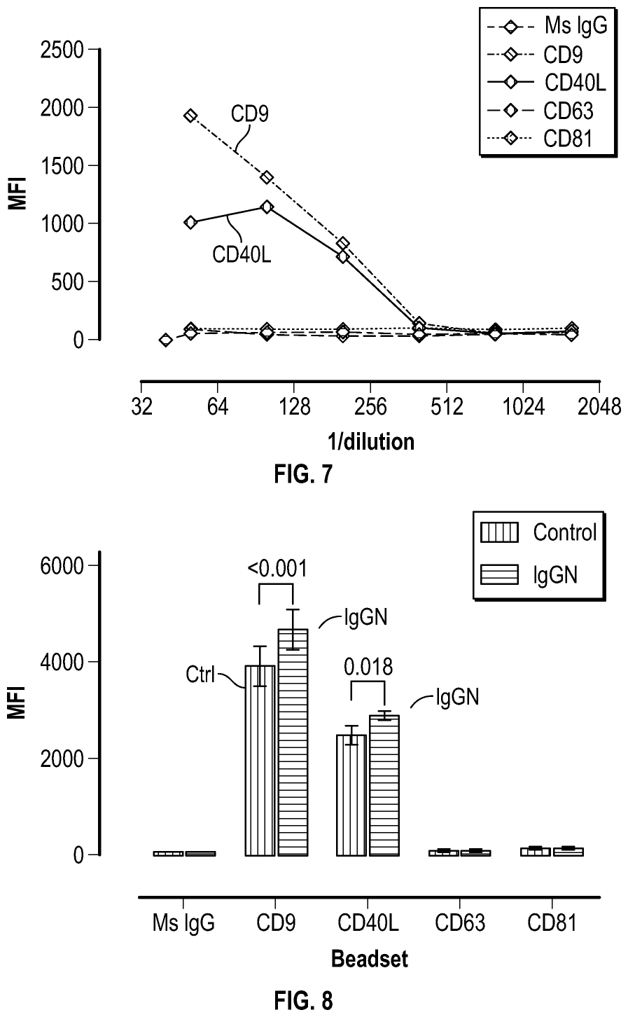

Luminex beads can identify Glomerulus-specific EV. As seen in FIG. 7: Rb-α-PODXL can be detected on EV containing CD9 or CD40L but not on EV containing CD63 or CD81; signal was found using 2 different α-PODXL antibodies; can only occur if both proteins are located on the same structure.

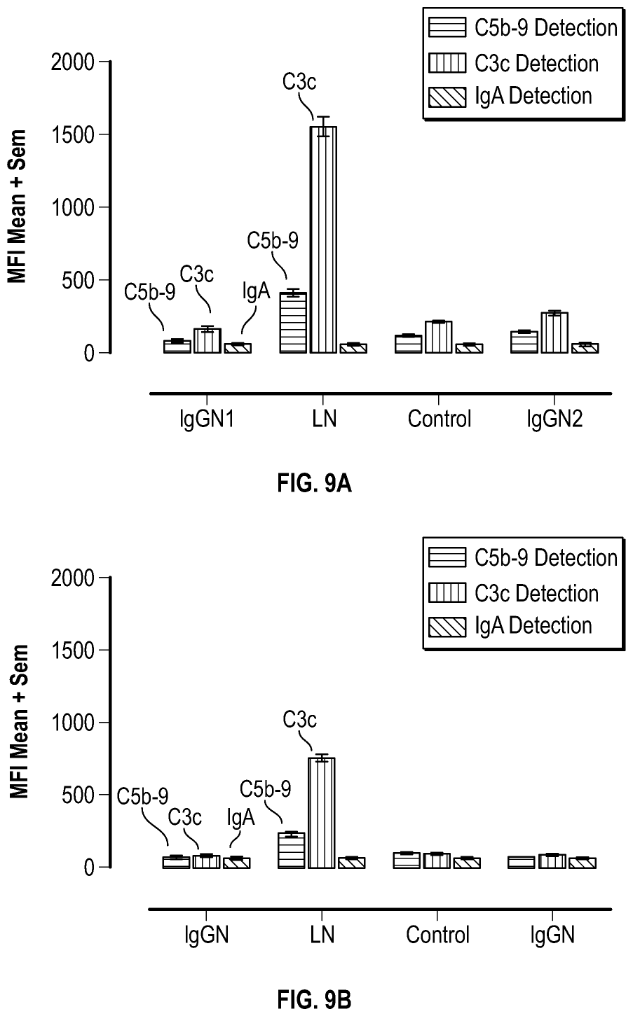

The results indicate that nephron-specific EV levels increase with disease. As seen in FIG. 8: there are more PODXL+ EV in IgAN urine than in Control urine; increases are seen in both CD9+/PODXL+ and CD40+/PODXL+ EV populations; and CD63+ and CD81+ EV are still negative.

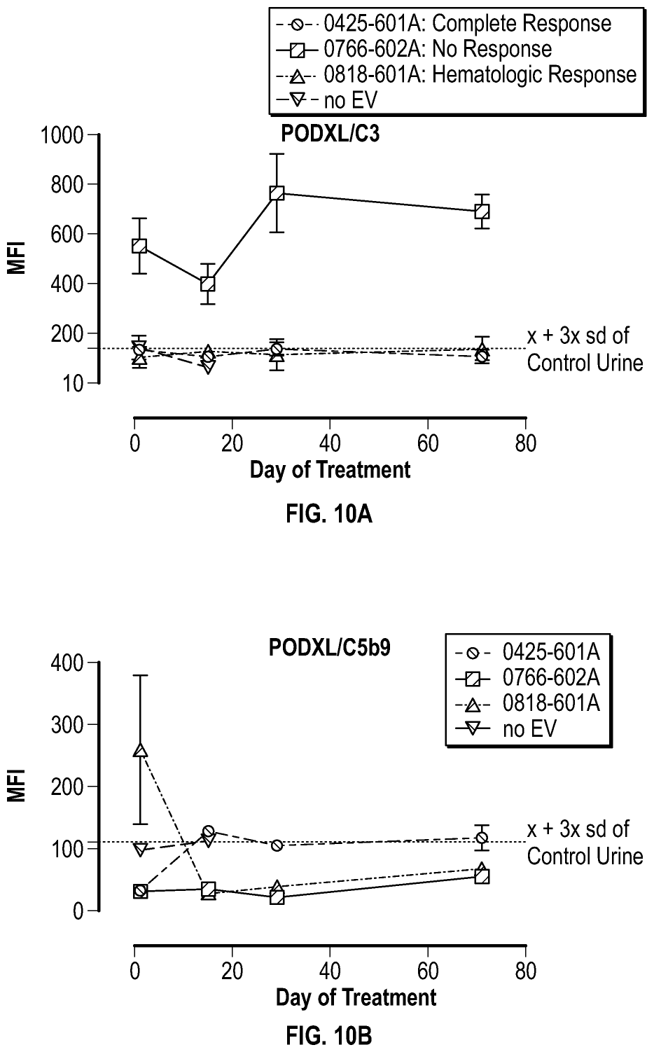

Luminex beads can measure complement on EV membranes. As seen in FIG. 9: C3c and C5b-9 are detected in LN patient urine on both the CD9+ and PODXL+ beads but not on CD63+ or CD81+; these results were confirmed in 10 LN, 6 IgAN, and 7 control samples (data not shown); both LN and IgAN samples can have C3, C5b-9, C4 and C1q deposition on both PODXL+ and AQP2+ EV compared to control samples.

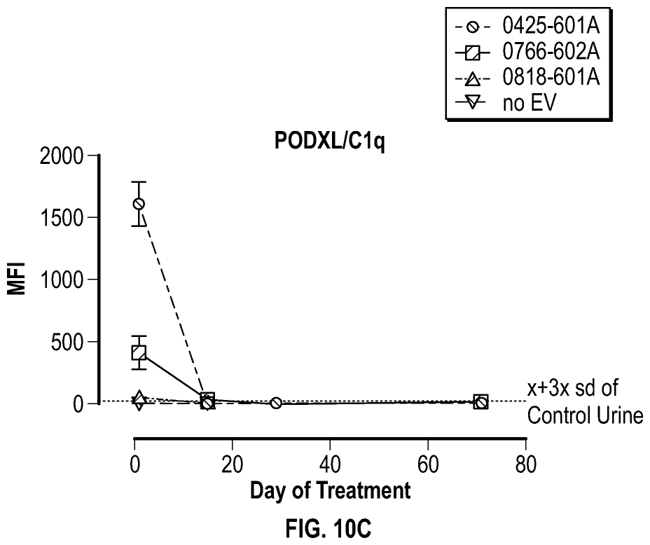

The results also indicate that glomerular C5b-9 deposition decreases with Ravulizumab treatment. As seen in FIG. 10: aHUS patients may have any combination of C3, C5b-9, C1q and C4 depositing on the podocyte membranes; C3 does not change during ravulizumab treatment; and C5b-9 and C1q levels on EV are rapidly reduced during ravulizumab treatment.

Kidney biopsy is the current standard for the differential diagnosis of chronic kidney disease. However, risk for serious complications limits patient selection and testing frequency. EV offer an easily accessible window to monitor ongoing complement deposition on renal membranes before and during treatment. It also allows precise cellular identification of complement attack along the nephron.

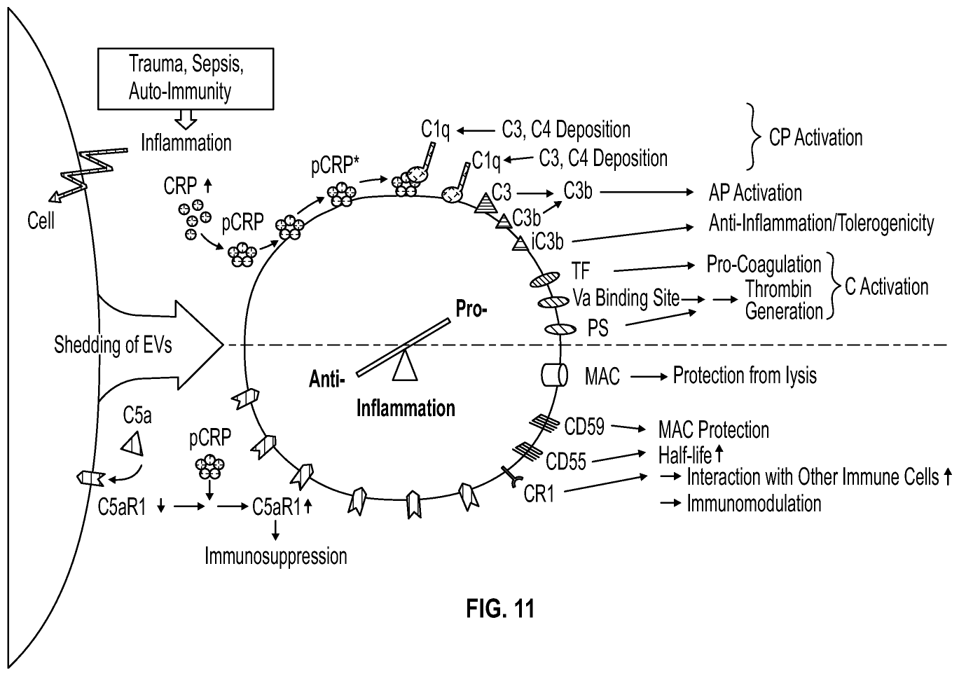

This technique can be expanded beyond kidney and urine to any biological sample. Any cell type or tissue of interest with a unique PM marker could be used to interrogate complement deposition. FIG. 11 provides a schematic representation of the markers that could be analyzed in accordance with the present disclosure.

### Example 3: Bioassay Using Modified-Membrane-Binding Fluorophores to Normalize EV Data

In order to improve assay robustness, it is important to measure the number of membrane vesicles isolated on each capture antibody-coated LUMINEX bead. In some aspects, a “normalization” step is implemented due to the variability in vesicles per bead and vesicles per mL of sample matrix within and between individual donors. Accordingly, it was contemplated that a modified membrane-binding fluorophore-cysteine-lysine-palmtoyl group (mCLING; Synaptic Systems, catalogue number 710-MCK) could improve the assay. In one aspect, mCLING was modified via biotinylation and the modified mCLING was added to each test sample in a replicate well of immune-bead captured EVs. After washing each bead set, Streptavidin Phycoerythrin (SA-PE) was added and fluorescent intensity was measured for the mCLING sample and the matching analyte sample.

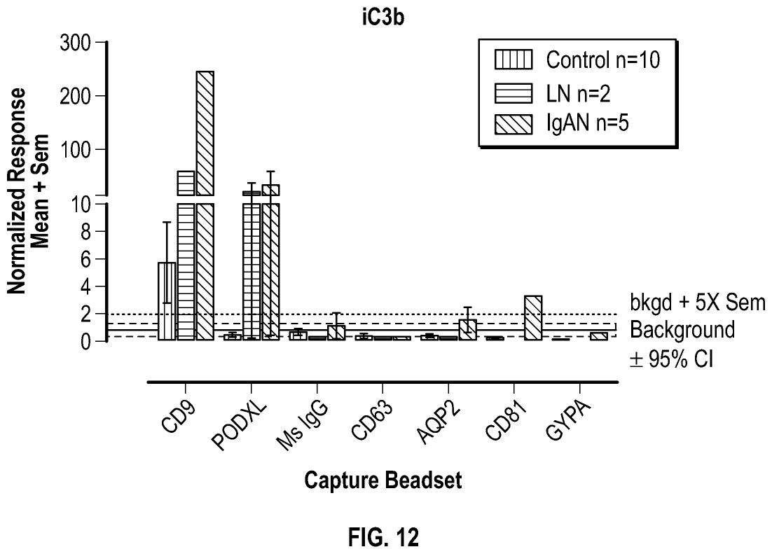

In one study, urine EVs were obtained from healthy volunteers (control) and lupus nephritis (LN) and IgA nephropathy (IgAN) patients, enriched with a panel of antibody capture beads and then surface phenotyped in duplicate wells for either (a) total EVs using biotinylated-mCLING or (b) complement iC3b using Quidel antibody A710 (monoclonal, neoantigen specific). The normalized signal for analyte (iC3b) was generated by taking a ratio of the iC3b signal and the signal for biotinylated-mCLING (signal=(b)/(a)). Results are shown in FIG. 12 which is a representative normalized EV data for iC3b. The normalized response (shown on the y-axis) represents mean fluorescence intensity value reported for each marker (as indicated in the x-axis) divided by their corresponding mCLING mean fluorescence intensity value.

The controls represents identical samples from healthy donors. Patients (LN or IgAN) are represented alongside for each marker.

The data show that certain EVs, e.g., those that are positive for CD9 and PODXL, selectively contain complement (e.g., iC3b) deposition above the background level (as indicated by the horizontal dashed line in FIG. 12). The data show that activated complement pathway proteins such as iC3b are associated with complement-mediated disease pathophysiology, as indicated in the EVs obtained from the two disease subsets, LN and IgAN, respectively.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries (e.g., PUBMED, NCBI or UNIPROT accession numbers), and other references mentioned herein are incorporated by reference in their entirety. In case of conflict, the present specification, including definitions, will control.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of detecting complement activity in a biological sample, from a subject, comprising:
(a) isolating a portion of the biological sample comprising extracellular vesicles (EV) or a membrane-bound portion thereof with at least one first capture antibody or an antigen-binding fragment thereof to capture at least one first marker on the EV or the membrane-bound portion thereof, wherein the first marker comprises an EV-specific marker or a tissue-specific marker displayed on the EV,
(b) optionally contacting a portion of the sample with at least one second capture antibody or an antigen-binding fragment thereof to capture at least one second marker on the EV or the membrane-bound portion thereof; and
(c) detecting, qualitatively or quantitatively, a presence or a level of a complement system-associated component on the captured EV or the membrane-bound portion thereof with at least one detection antibody or antigen-binding fragment thereof which is specific for the complement system-associated component, thereby detecting complement activation in the biological sample.

2. The method of claim 1, wherein the first capture marker comprises an EV-specific marker and the optional second capture marker comprises a tissue-specific marker displayed on the EVs or the membrane-bound portion thereof.

3. The method of claim 1 or 2, wherein both the first capture marker and the second capture marker are present, and the first capture marker comprising an EV-specific marker and the second capture marker comprising a tissue-specific marker are detected.

4. The method of any of claims 1-3, wherein the biological sample is from a tissue, an organ, or a body fluid.

5. The method of any of claims 1-4, wherein the biological sample comprises EVs or membrane-bound portions thereof from bladder cells, kidney cells, whole blood, red blood cells, platelets, serum, plasma, a blood fraction other than serum or plasma, lymph, cerebrospinal fluid (CSF), saliva, tears, vaginal discharge, semen, glandular secretions, exudate, contents of cysts or feces, lavage, or ascites.

6. The method of any one of claims 1-5, wherein the biological sample comprises EVs or membrane-bound portions thereof from glomerular podocytes, convoluted tubule of the kidney, or bladder epithelium; or red blood cells (RBC).

7. The method of any one of claims 1-6, wherein the first capture antibody or the antigen-binding fragment thereof is conjugated to a first solid support, optionally the second capture antibody or the antigen-binding fragment thereof is conjugated to a second solid support, and the detection antibody is conjugated to a detectable marker.

8. The method of claim 7, comprising contacting a portion of the biological sample with the first capture antibody or an antigen-binding fragment thereof and the second capture antibody or an antigen-binding fragment thereof, wherein the first capture antibody or the antigen-binding fragment thereof and the second capture antibody or the antigen-binding fragment thereof are conjugated to the same support or to different supports.

9. The method of claim 7, wherein the detectable marker is selected from the group consisting of: fluorophores, chromogens, and biotin.

10. The method of any one of claims 7-9, wherein the detectable marker is a fluorophore with an absorption maximum between 500-1000 nm, and emission maximum between 550-1100 nm.

11. The method of any one of claims 7-10, wherein the detectable marker is phycoerythrin (PE).

12. The method of any one of claims 7-10, wherein the detectable marker is biotin.

13. The method of any one of claims 7-12, wherein the first and second solid supports are independently selected from the group consisting of: nanoparticles, microparticles, beads, magnetic beads, nanostructures, tissue culture plate, silica, and nanomatrices.

14. The method of any one of claims 1-13, wherein:
the first marker is selected from the group consisting of extracellular vesicle-associated proteins;
optionally, the second marker is selected from the group consisting of tissue-specific extracellular vesicle-associated proteins; and
the complement system-associated component is selected from the group consisting of: (a) a component of the alternative pathway (AP), (b) a component associated with classical pathway (CP), and (c) a component associated with lectin pathway (MBL).

15. The method of any of claims 1-14, wherein the complement system-associated component is selected from the group consisting of (a) a component of the alternative pathway (AP) and (b) a component associated with classical pathway (CP).

16. The method according to claim 14, wherein the complement system-associated component is a protein selected from the group consisting of C3, C5b-9, C4, C1q, C9, C3b, iC3b, TF, CRP, pCRP, MAC, CD59, CD55, CR1, C5aR1, and C5a.

17. The method of any one of claims 1-16, wherein:
the first marker is selected from the group consisting of: ALIX, TSG101, CD9, CD63, CD81, CD40L, CD26, CD31, CD45, CD2, CD11a, CD24, CD55, CD59, CF106, CD56, CD51, CD82, Integrins, Tetraspanins, Annexins, HSP90, HSP70, Syntenin-1, ADAM10, EHD4, Actin, Rab5, clathrin, Flotillin-1, MHC I, MHC II, Actinin-4, GP96, EHD4, Mitofilin, and LAMP2;
the second marker is selected from the group consisting of: podocalyxin (PODXL), aquaporin 2 (AQP 2), uroplakin1b (UPK1b), podocin (NPHS2), glycophorin A (GYPA), mucin-1, type 2 Na-K-2Cl co-transporter (NKCC2), aquaporin 1 (AQP 1), α-glutathione-S-transferase (alpha-GST), Tamm-Horsfall protein (TH), calbindin-D28K (CalD), megalin, cubilin, nephrin (Nphs1), Claudin-1, Annexin-V, synaptopodin (Synpo), Wilm's tumor protein (WU), Band 3, stomatin (STOM), BGP1, Globin, Glycophorin B, Rh polypeptides, and Rh glycoprotein; and
the complement protein is selected from the group consisting of: MAC, C3, C5b-9, C4, C1q, and C9.

18. The method of claim 17, wherein the biological sample comprises EVs from kidney cells and the second marker is a kidney-specific EV marker selected from the group consisting of podocalyxin (PODXL), aquaporin 2 (AQP 2), uroplakin1b (UPK1b) and podocin (NPHS2).

19. The method of claim 17, wherein the sample comprises EVs from red blood cells (RBC) and the second marker is an RBC-specific EV marker selected from glycophorin A (GYPA).

20. The method of claim 17, wherein the sample comprises EVs which is negative for CD81 as a first marker and/or Uroplakin 1B (UPK1B) as a second marker.

21. The method of claim 1, wherein the capture and detection markers are present in the same EV or a membrane-bound portion thereof.

22. The method of any one of claims 1-21, further comprising determining if the subject suffers from a complement-mediated disease or is at risk of developing a complement-mediated disease, comprising comparing the presence or level of the component of the complement pathway on the EV or the membrane-bound portion thereof to a control.

23. The method of claim 22, wherein the control comprises an identical sample from a healthy subject.

24. The method of claim 22 or 23, comprising indicating that the subject suffers from a complement-mediated disease or is at risk of developing a complement-mediated disease if: the level or the presence of the component of the complement pathway on the EVs or the membrane-bound portion thereof obtained from the subject is enhanced compared to the control.

25. A method for diagnosis or prognostic assessment of a complement-mediated disease in a subject, comprising:
(a) obtaining a sample comprising extracellular vesicles (EV) or a membrane-bound portion thereof from the subject,
(b) contacting a portion of the sample with at least one first capture antibody or an antigen-binding fragment thereof to capture at least one first marker on the EVs or the membrane-bound portion(s) thereof;
(c) optionally contacting a portion of the sample with at least one second capture antibody or an antigen-binding fragment thereof to capture at least one second marker on the EVs or the membrane-bound portion(s) thereof;
(d) contacting the captured EVs or membrane-bound portions thereof comprising the at least one first marker and optionally the at least one second marker with at least one detection antibody or an antigen-binding fragment thereof specific for a complement system-associated component; and
(e) detecting, qualitatively or quantitatively, the detection antibody or the antigen-binding fragment thereof, to measure, a presence of or a level of the component of the complement pathway on the EV or the membrane-bound portion thereof, wherein an elevation in the presence or level of the component of the complement pathway in the subject's sample compared to a control indicates the subject has or is at risk of developing the complement-mediated disease.

26. The method of claim 25, wherein the first marker comprises an EV-specific marker or a tissue-specific marker displayed on EV.

27. The method of claim 25, wherein the first marker comprises an EV-specific marker and the second marker comprises a tissue-specific marker displayed on EVs.

28. A method for monitoring response to treatment of a complement-mediated disease with a complement modulator in a subject, comprising:
(a) obtaining a sample comprising extracellular vesicles (EV) or a membrane-bound portion thereof from the subject before and after the treatment,
(b) contacting a portion of the sample with at least one first capture antibody or an antigen-binding fragment thereof to capture at least one first marker on the EVs or the membrane-bound portion(s) thereof;
(c) optionally contacting a portion of the sample with at least one second capture antibody or an antigen-binding fragment thereof to capture at least one second marker on the EVs or the membrane-bound portion(s) thereof;
(d) contacting the captured EVs or membrane-bound portions thereof comprising the at least one first marker and optionally the at least one second marker with at least one detection antibody or an antigen-binding fragment thereof specific for a complement system-associated component; and
(e) detecting, qualitatively or quantitatively, the detection antibody or the antigen-binding fragment thereof, to measure, a presence of or a level of the component of the complement pathway on the EV or the membrane-bound portion thereof, wherein an attenuation in the presence or level of the component of the complement pathway in the subject's sample after treatment with the complement modulator compared to before treatment with the complement modulator indicates the subject is responding to the complement modulator.

29. The method of claim 28, wherein the complement mediator is a complement 5 (C5) inhibitor, complement 5a (C5a) inhibitor, complement 5 receptor (C5R1) inhibitor, complement 3 (C3) inhibitor, Factor D (FD) inhibitor, Factor H (FH) inhibitor, Factor B (FB) inhibitor, MASP2 inhibitor, MASP3 inhibitor, properdin inhibitor or a combination thereof.

30. The method of any of claims 25-29, wherein the disease is an inflammatory disease or a thrombotic disease.

31. The method according to any of claims 25-29, wherein the disease is a thrombotic hematological disease or a thrombotic nephrological disease.

32. The method of any of claims 25-29, wherein the disease is a nephrological disease selected from the group consisting of atypical haemolytic uraemic syndrome (aHUS), C3 glomerulopathy (C3G), dense deposit disease (DDD), membranoproliferative glomerular nephritis (MPGN), lupus nephritis (LN), IgA nephropathy (IN), lupus nephritis (LN), membranous nephropathy (MN), complications due to hemodialysis in transplant patients, antibody-mediated rejection (AMR) and anti-neutrophil cytoplasmic antibody (ANCA)-associated vasculitis (AAV).

33. The method of any of claims 25-29, wherein the disease is a hematological disease selected from the group consisting of paroxysmal nocturnal hemoglobinuria (PNH), atypical haemolytic uraemic syndrome (aHUS), secondary HUS due to solid organ transplant or hematopoietic stem cell transplants, Thrombotic microangiopathy (TMA), and cold agglutinin disease (CAD).

34. The method according to any of claims 1-33, wherein the detection comprises immunoassay (e.g., ELISA or RIA), electron microscopy (EM), tandem mass tag (TMT), a luminescence assay (e.g., LUMINEX), or fluoroimmnoassay (FIA).

35. The method according to any of claims 1-34, wherein detection step is carried out in a multiplex format.

36. The method according to any of claims 1-34, wherein detection step is carried out by measuring markers in several discrete tissues in one sample and/or monitoring multiple potential complement proteins & pathways in a single assay.

37. The method according to any of claims 1-36, wherein an EV enrichment step is carried out before the first marker and optionally the second marker is contacted with the respective antibody or the antigen-binding fragment thereof.

38. The method according to any of claims 1-37, wherein the biological sample is procured from a non-invasive, liquid biopsy protocol.

39. A method of detecting complement activation in a subject's kidney tissue, comprising:
(a) contacting the subject's urine sample comprising extracellular vesicles (EV) or a membrane-bound portion thereof comprising a first marker which is an EV-specific marker or a tissue-specific marker displayed on EVs or membranes thereof with a first capture antibody or an antigen-binding fragment thereof specific thereto, thereby capturing EVs or membranes containing the first marker;
(b) optionally contacting the sample with a second capture antibody or an antigen-binding fragment thereof to capture EVs or membrane-bound portion(s) thereof comprising the second capture marker which is different from the first marker; and
(c) detecting, qualitatively or quantitatively, a presence or a level of the component of the complement pathway on the captured EV or the membrane-bound portion thereof with an antibody or an antigen-binding fragment thereof specific to the component, thereby detecting complement activation in the biological sample;
wherein the EV-specific marker is selected from the group consisting of CD9, CD63, and CD81,
the tissue-specific marker is selected from the group consisting of:
(1) podocalyxin (PODXL) specific for podocytes in the glomerulus;
(2) aquaporin 2 (AQP2) specific for the convoluted tubule epithelium;
(3) uroplakin1b (UPK1b) specific for bladder epithelium; and
(4) glycophorin A (GYPA) specific for red blood cells (RBC), and
the component of the complement pathway is selected from the group consisting of MAC, C3, C5b-9, C4, C1q, and C9.

40. Use of: at least one first capture antibody to capture at least one first target; at least one second capture antibody to capture at least one second target; and at least one detection antibody specific for a complement protein to detect an amount of the captured at least one first target, the captured at least one second target, or both.

41. A method of screening a test compound for complement modulation, comprising
(a) obtaining a sample containing extracellular vesicles (EV) or a membrane-bound portion thereof from a subject suffering from a complement-mediated disease (e.g., an animal such as a mouse, rabbit, hamster, sheep, llama, dog, monkey, chimpanzee or human) before and after administering the subject with the test compound;
(b) contacting a portion of the sample with at least one first capture antibody or an antigen-binding fragment thereof to capture at least one first marker on the EVs or the membrane-bound portion(s) thereof;
(c) optionally contacting a portion of the sample with at least one second capture antibody or an antigen-binding fragment thereof to capture at least one second marker on the EVs or the membrane-bound portion(s) thereof;
(d) contacting the captured EVs or membrane-bound portions thereof with at least one detection antibody or an antigen-binding fragment thereof specific for a complement system-associated component; and
(e) detecting, qualitatively or quantitatively, the detection antibody or the antigen-binding fragment thereof, to measure, a presence of or a level of the complement component on the EV or the membrane-bound portion thereof, wherein a modulation (e.g., increase or decrease; preferably decrease) in the presence or level of the complement component in the subject's sample after administration of the test compound compared to before administration of the test compound indicates that the test compound is capable of complement modulation.

42. The method of claim 41, wherein the test compound is specifically capable of modulating C1q, C1, C1s, C2, MASP-2, MASP-3, Factor D, Factor B, Properdin (Factor P), Factor H, C3/C5 Convertase, C5, C5a/C5aR, C3a/C3aR, C6, or CD59.

43. The method of claim 41, wherein the test compound is a monoclonal antibody or small molecule or siRNA/RNAi.

44. The method of claim 41, wherein the modulating activity of the test compound is compared to the modulating activity of a molecule having complement modulating activity.

45. The method of claim 44, wherein the molecule is provided in Table A.
